(19) United States
(12) Patent Application Publication
(10) Pub. No.: US 2010/0311688 A1
(43) Pub. Date: Dec. 9, 2010

(54) OPHTHALMIC FORMULATIONS, METHODS OF MANUFACTURE, AND METHODS OF USING SAME

(75) Inventors: Matthew J. Chapin, Amesbury, MA (US); George Minno, Suwanee, GA (US); Jackie Nice, Medford, MA (US); George W. Ousler, III, North Andover, MA (US); Mark B. Abelson, Andover, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Aciex Therapeutics, Inc., Westborough, MA (US)

(21) Appl. No.: 12/794,315

(22) Filed: Jun. 4, 2010

Related U.S. Application Data
(60) Provisional application No. 61/184,435, filed on Jun. 5, 2009.

(51) Int. Cl. A61K 31/717; A61P 27/04

(52) U.S. Cl. 514/57

(57) ABSTRACT

The present invention provides compositions for treating and/or preventing signs and symptoms associated with dry eye and/or ocular irritation, and methods of use thereof. Such compositions are provided in novel ophthalmic formulations that are comfortable upon instillation in the eye. Methods of manufacture are also provided.

### REFERENCE TO RELATED APPLICATIONS

This application claims priority under 35 U.S.C. §119(e) to U.S. Provisional Application Ser. No. 61/184,435, filed Jun. 5, 2009, the contents of which are hereby incorporated by reference in its entirety.

### FIELD OF THE INVENTION

The invention relates generally to ophthalmic formulations useful for the treatment of ocular disorders, such as for example dry eye disease, and more particularly to ophthalmic formulations comprising a tear substitute, or one or more components thereof. The invention further relates to methods of manufacture and methods of using the ophthalmic formulations of the invention for the treatment of ocular disorders, such as for example, dry eye disease.

### BACKGROUND OF THE INVENTION

Dry eye disease is an ocular disease affecting approximately 10-20% of the population. This disease progressively affects larger percentages of the population as it ages, with the majority of these patients being women. In addition, almost everyone experiences ocular irritation, or the symptoms and/or signs of dry eye as a condition, from time to time under certain circumstances, such as prolonged visual tasking (e.g. working on a computer), being in a dry environment, using medications that result in ocular drying, etc.

In individuals suffering from dry eye, the protective layer of tears that normally protects the ocular surface is compromised, a result of insufficient or unhealthy production of one or more tear components. This can lead to exposure of the surface of the eye, ultimately promoting desiccation and damage of surface cells. Signs and symptoms of dry eye include but are not limited to keratitis, conjunctival and corneal staining, redness, blurry vision, decreased tear film break-up time, decreased tear production, tear volume, and tear flow, increased conjunctival redness, excess debris in the tear film, ocular dryness, ocular grittiness, ocular burning, foreign body sensation in the eye, excess tearing, photophobia, ocular stinging, refractive impairment, ocular sensitivity, and ocular irritation. Patients may experience one or more of these symptoms. The excess tearing response may seem counterintuitive, but it is a natural reflex response to the irritation and foreign body sensation caused by the dry eye. Some patients may also experience ocular itching due to a combination of ocular allergy and dry eye symptoms.

There are many possible variables that can influence a patient's signs or symptoms of dry eye including levels of circulating hormones, various autoimmune diseases (e.g. Sjögren's syndrome and systemic lupus erythematosus), ocular surgeries including PRK or LASIK, many medications, environmental conditions, visual tasking such as computer use, ocular fatigue, contact lens wear, and mechanical influences such as corneal sensitivity, partial lid closure, surface irregularities (e.g. pterygium), and lid irregularities (e.g. ptosis, entropion/ectropion, pinguecula). Environments with low humidity, e.g., those that cause dehydration, can exacerbate or cause dry eye symptoms, such as sitting in a car with the defroster on or living in a dry climate zone. In addition, visual tasking can exacerbate symptoms. Tasks that can greatly influence symptoms include watching TV or using a computer for long periods of time where the blink rate is decreased.

It is therefore an object of the present invention to provide ophthalmic formulations/compositions that are useful for treating the signs and symptoms of dry eye disease. It is a further object of the present invention to provide ophthalmic formulations/compositions that are useful as a moisturizing and lubricating eye drop (i.e., an artificial tear solution), a delivery vehicle for ophthalmic drugs, and/or as a contact lens wetting and lubricating solution. It is even a further object of the present invention to provide more efficient methods of manufacturing ophthalmic formulations than those currently employed or known.

### SUMMARY OF THE INVENTION

The ophthalmic formulations of the present invention are useful for treating the signs and symptoms of dry eye disease, more specifically for improving tear film stability (i.e., increasing tear film break up time (TFBUT), reducing corneal staining and reducing ocular discomfort. Treatment of the signs and symptoms of dry eye is provided by protection of the ocular surface via enhancement of the tear film. One measure of tear film stability is increased tear film break up time (TFBUT). One method of determining a clinically meaningful increase in TFBUT is an increase in Ocular Protection Index (OPI).

The ophthalmic formulations of the present invention are characterized by 1) a novel, optimal viscosity which coats the ocular surface better than prior artificial tear formulations known in the art (no clumping), leading to longer dwell time/retention time and thereby enhanced ocular surface protection, and 2) hypo-tonicity, addressing the underlying hyper-tonic conditions characterized by dry eye, in a single physiologically-based, preservative-free eye-drop solution. According to preferred embodiments, there is provided an aqueous ophthalmic solution containing a viscosity enhancing agent or combination of viscosity enhancing agents, a tonicity agent or combination of tonicity agents, and a buffer or combination of buffers. The viscosity enhancing agent is preferably hydroxypropyl methylcellulose (also commonly referred to as hypromellose or HPMC), and in particular Methocel® E4M Premium—Hydroxypropyl methylcellulose. Preferably, the ophthalmic formulations of the invention contain 0.72% to 0.8% hydroxypropyl methylcellulose and are manufactured in accordance with the methods described herein.

For example, the ophthalmic formulation of the invention is manufactured by: heating a solution base comprising water to at least 85° C.; mixing the solution base at a rate of at least 10 rotations per minute; adding 0.72% to 0.8% hydroxypropyl methylcellulose at a rate of at least 0.1 gm/min until a target viscosity is achieved; wherein the target viscosity is from about 70 to 88 cpi. Optionally, one or more tonicity agents including but not limited to NaCl, KCl, ZnCl₂, CaCl₂, and MgCl₂ are added in a ratio that provides an osmolality range of about 260 to about 300 mOsm/kg. A buffer system such as borate buffers, phosphate buffers, citrate buffers, and combinations and mixtures thereof may also be added.

In one particular embodiment, the ophthalmic formulation of the invention is a 0.8% hypromellose solution comprised of the following: 8.0 mg/ml Hypromellose, USP Type 2910 (Methocel® E4M Premium—Hydroxypropyl methylcellulose); 8.0 mg/ml sodium chloride, USP; 0.15 mg/ml edetate sodium, USP; Sodium hydroxide, 5 N or Hydrochloric Acid, 5 N to adjust pH to 7.4; and purified water, USP (QS to 1 ml). Optionally, this 0.8% hypromellose solution contains a preservative such as benzalkonium chloride or a derivative thereof (e.g., Polyquad®), a stabilized oxychloro complex (e.g., Purite®), sodium perborate, or sorbate. Preferably, this 0.8% hypromellose solution is formulated at a viscosity of approximately 70-88 cpi.

In another particular embodiment, the ophthalmic formulation of the invention is a 0.8% hypromellose solution formulated for unit dose comprised of the following: 8.0 mg/ml Hypromellose, USP Type 2910 (Methocel® E4M Premium—Hydroxypropyl methylcellulose); 10 mg/ml glycerin, USP; 4.8 mg/ml sodium chloride, USP; 0.8 mg/ml potassium chloride, USP; 0.08 mg/ml calcium chloride, USP; 0.05 mg/ml magnesium chloride, USP; 1.7 mg/ml sodium citrate, USP; NaOH/HCl to adjust pH to 7.4; and purified water, USP (QS to 1 ml). Optionally, this 0.8% hypromellose solution contains a preservative such as benzalkonium chloride or a derivative thereof (e.g., Polyquad®), a stabilized oxychloro complex (e.g., Purite®), sodium perborate, or sorbate. Preferably, this 0.8% hypromellose unit dose solution is formulated at a viscosity of 70-88 cpi.

In still another particular embodiment, the ophthalmic formulation of the invention is a 0.8% hypromellose solution formulated for multi-dose use comprised of the following: 8.0 mg/ml Hypromellose, USP Type 2910 (Methocel® E4M Premium—Hydroxypropyl methylcellulose); 10 mg/ml glycerin, USP; 4.8 mg/ml sodium chloride, USP; 0.8 mg/ml potassium chloride, USP; 0.08 mg/ml calcium chloride, USP; 0.05 mg/ml magnesium chloride, USP; 5.0 mg/ml boric acid, NF; 1.0 mg/ml sorbic acid, NF; 1.0 mg/ml disodium edetate, USP; NaOH/HCl to adjust pH to 7.4; and purified water, USP (QS to 1 ml). Optionally, this 0.8% hypromellose solution contains a preservative such as benzalkonium chloride or a derivative thereof (e.g., Polyquad®), a stabilized oxychloro complex (e.g., Purite®), sodium perborate, or sorbate. Preferably, this 0.8% hypromellose multi-dose solution is formulated at a viscosity of 70-88 cpi.

The ophthalmic formulations/compositions of the present invention find particular utility as a treatment for the signs and symptoms of dry eye disease, in addition to utility as a moisturizing and lubricating eye drop (i.e., an artificial tear solution), a delivery vehicle for ophthalmic drugs, and as a contact lens wetting and lubricating solution. An effective amount of the formulations may be used to treat and/or prevent signs and symptoms associated with dry eye disease and/or general eye irritation, and can also be used to treat another eye disorder if it contains a drug for that disorder. Thus, according to preferred embodiments, the aqueous ophthalmic formulations/compositions of the present invention are suitable for use as artificial tear products to relieve symptoms of dry eye disease.

Signs and/or symptoms associated with dry eye disease include but are not limited to ocular discomfort including but not limited to stinging, itching, burning, scratchiness and/or foreign body sensation in the eye(s); stringy mucus in or around the eye(s); eye redness; increased eye irritation from smoke and/or wind; eye fatigue after periods of reading or watching television; sensitivity to light; difficulty wearing contact lenses; a decrease in tear film integrity; an increase in corneal staining; blurred vision that improves with blinking; excessive tearing; or any combination thereof. The ophthalmic formulations provided herein are capable of providing immediate relief from the signs and symptoms of dry eye (as evidenced by decreased corneal staining, increased tear film break up time (TFBUT) and ocular protection index (OPI) (i.e., signs of dry eye), and reduction of ocular discomfort (i.e., symptoms) and are suitable for intermittent and/or repeated long term use for the treatment of acute or chronic dry eye disease either alone, or in conjunction with other concomitant therapies.

According to preferred embodiments, the ophthalmic formulations of the present invention are suitable for the treatment of dry eye disease. According to preferred embodiments, the ophthalmic formulations of the present invention relieve ocular discomfort and prolong the integrity of the tear film, as evidenced by an increase in tear film break up time (TFBUT). Alternatively, the compositions of the present invention may act as a vehicle for an ophthalmic drug.

An additional feature is the method of manufacture of a comfortable ophthalmic formulation for instillation into the eye, wherein said formulation comprises one or more tear substitute components.

Further, featured are kits for the shipping, storage or use of the formulations, as well the practice of the methods. Other features and advantages of the invention will become apparent from the following detailed description and claims.

### BRIEF DESCRIPTION OF THE DRAWINGS

FIG. 1 is a line graph depicting the efficacy of an ophthalmic formulation comprising 0.8% hydroxypropyl methylcellulose (HPMC) (designated as “AC-111”) on improving tear film stability (i.e., increasing tear film break-up time or “TFBUT”) in subjects exposed to a controlled adverse environment (CAE) chamber, as compared to a vehicle (i.e., no HPMC) control.

FIG. 2 is a line graph depicting a comparison of the mean efficacy of an ophthalmic formulation comprising 0.8% HPMC/0.3% ketorlac (w/v), 0.3% ketorolac alone (w/v), or vehicle control (i.e., no HPMC) on reducing corneal staining in subjects exposed to a controlled adverse environment (CAE) chamber. For each clustering of 3 bars shown on the graph, the 0.8% HPMC/0.3% ketorolac (w/v) formulation is represented by the bar on the far left, the 0.3% ketorolac alone (w/v) formulation is represented by the bar in the middle, and the vehicle alone formulation is represented by the bar on the far right.

FIG. 3 is a line graph depicting the efficacy of an ophthalmic formulation comprising 0.8% hydroxypropyl methylcellulose (HPMC) formulation in relieving ocular discomfort over a 42 day period in subjects having dry eye disease as compared to vehicle (i.e., no HPMC) control.

FIG. 4 is a line graph depicting the mean efficacy of an ophthalmic formulation comprising 0.8% hydroxypropyl methylcellulose (HPMC) (designated as AC-111) in relieving ocular discomfort at specific timepoints throughout each day (morning, afternoon, evening, bedtime, following morning) over the course of 42 days in subjects having dry eye disease, as compared to vehicle (i.e., no HPMC) control. For each cluster of bars shown on the graph at each timepoint, the AC-111 treatment group is represented by the bar on the left, the vehicle treatment group is represented by the bar on the right.

FIG. 5 is a bar graph depicting the mean change from baseline in tear film break up time (TFBUT) after treatment with an ophthalmic formulation comprising HMPC and 0.25% ketorolac formulated at a viscosity of 70 cpi in subjects exposed to a controlled adverse environment (CAE) chamber.

FIG. 6 is a bar graph depicting the mean change from baseline in the ocular protection index (OPI) after treatment with an ophthalmic formulation comprising HMPC and 0.25% ketorolac formulated at a viscosity of 70 cpi in subjects exposed to a controlled adverse environment (CAE) chamber.

FIG. 7 is a bar graph depicting the mean lid caking score (0=no lid caking; 1=lid caking) after treatment with an ophthalmic formulation comprising HMPC and 0.25% ketorolac formulated at a viscosity of 70 cpi in subjects exposed to a controlled adverse environment (CAE) chamber.

FIG. 8 is a bar graph depicting the mean blurring score (0=no blurring; 1=blurring) after treatment with an ophthalmic formulation comprising HMPC and 0.25% ketorolac formulated at a viscosity of 70 cpi in subjects exposed to a controlled adverse environment (CAE) chamber.

FIG. 9 is a bar graph depicting the mean comfort score (0=very comfortable/best, 10=very uncomfortable/worst) after treatment with an ophthalmic formulation comprising HMPC and 0.25% ketorolac formulated at a viscosity of 70 cpi in subjects exposed to a controlled adverse environment (CAE) chamber.

FIG. 10 is a bar graph depicting the mean change from baseline in tear film break up time (TFBUT) after treatment with an ophthalmic formulation comprising HMPC and 0.25% ketorolac formulated at a viscosity of 90 cpi in subjects exposed to a controlled adverse environment (CAE) chamber.

FIG. 11 is a bar graph depicting the mean change from baseline in the ocular protection index (OPI) after treatment with an ophthalmic formulation comprising HMPC and 0.25% ketorolac formulated at a viscosity of 90 cpi in subjects exposed to a controlled adverse environment (CAE) chamber.

FIG. 12 is a bar graph depicting the mean lid caking score (0=no lid caking; 1=lid caking) after treatment with an ophthalmic formulation comprising HMPC and 0.25% ketorolac formulated at a viscosity of 90 cpi in subjects exposed to a controlled adverse environment (CAE) chamber.

FIG. 13 is a bar graph depicting the mean blurring score (0=no blurring; 1=blurring) after treatment with an ophthalmic formulation comprising HMPC and 0.25% ketorolac formulated at a viscosity of 90 cpi in subjects exposed to a controlled adverse environment (CAE) chamber.

FIG. 14 is a bar graph depicting the mean comfort score (0=very comfortable/best, 10=very uncomfortable/worst) after treatment with an ophthalmic formulation comprising HMPC and 0.25% ketorolac formulated at a viscosity of 90 cpi in subjects exposed to a controlled adverse environment (CAE) chamber.

FIG. 15 is a bar graph depicting the mean change from baseline in tear film break up time (TFBUT) after treatment with an ophthalmic formulation comprising HMPC and 0.25% ketorolac formulated at a viscosity of 120 cpi in subjects exposed to a controlled adverse environment (CAE) chamber.

FIG. 16 is a bar graph depicting the mean change from baseline in the ocular protection index (OPI) after treatment with an ophthalmic formulation comprising HMPC and 0.25% ketorolac formulated at a viscosity of 120 cpi in subjects exposed to a controlled adverse environment (CAE) chamber.

FIG. 17 is a bar graph depicting the mean lid caking score (0=no lid caking; 1=lid caking) after treatment with an ophthalmic formulation comprising HMPC and 0.25% ketorolac formulated at a viscosity of 120 cpi in subjects exposed to a controlled adverse environment (CAE) chamber.

FIG. 18 is a bar graph depicting the mean blurring score (0=no blurring; 1=blurring) after treatment with an ophthalmic formulation comprising HMPC and 0.25% ketorolac formulated to have a viscosity of 120 cpi in subjects exposed to a controlled adverse environment (CAE) chamber.

FIG. 19 is a bar graph depicting the mean comfort score (0=very comfortable/best, 10=very uncomfortable/worst) after treatment with an ophthalmic formulation comprising HMPC and 0.25% ketorolac formulated to have a viscosity of 120 cpi in subjects exposed to a controlled adverse environment (CAE) chamber.

### DETAILED DESCRIPTION

The ophthalmic formulations of the present invention provide ocular surface protection via enhancement of the tear film (as evident by increased tear film break up time). According to preferred embodiments, there is provided aqueous ophthalmic solutions containing a viscosity enhancing agent or combination of viscosity enhancing agents, a tonicity agent or combination of tonicity agents, and a buffer or combination of buffers. Effective amount of these formulations may be used to treat and/or prevent signs and symptoms associated with dry eye and/or general eye irritation, and can also be used to treat another eye disorder if it contains a drug for that disorder.

The extraordinary efficacy of the ophthalmic formulations of the invention is attributed to, among other things, the synergistic effect of the combination of ingredients and the optimal viscosity achieved by the method of manufacture described herein. The combination of ingredients and optimal viscosity achieved by the method of manufacture of the tear substitute component(s) provides an ophthalmic formulation which covers the ocular surface better than prior artificial tear solutions known in the art (i.e., the formulations of the present invention coat the ocular surface without clumping), leading to an increased retention time on the ocular surface over prior artificial tear formulations of varying viscosity, thereby enhancing the integrity of the tear film and providing protection of the ocular surface (e.g., as evidenced by increased tear film break-up time and/or the ocular protection index). As such, the comfortable ophthalmic formulations described herein will treat signs and symptoms of dry eye, increase patient compliance in the use of such formulations for the treatment and/or prevention of signs and symptoms associated with dry eye disease and/or ocular discomfort.

### Viscosity Enhancing Agents

A variety of viscosity enhancing agents are known in the art and include, but are not limited to: polyols such as, glycerol, glycerin, polyethylene glycol 300, polyethylene glycol 400, polysorbate 80, propylene glycol, and ethylene glycol, polyvinyl alcohol, povidone, and polyvinylpyrrolidone; cellulose derivatives such hydroxypropyl methylcellulose (also known as hypromellose and HPMC), carboxymethyl cellulose sodium, hydroxypropyl cellulose, hydroxyethyl cellulose, and methyl cellulose; dextrans such as dextran 70; water soluble proteins such as gelatin; carbomers such as carbomer 934P, carbomer 941, carbomer 940 and carbomer 974P; and gums such as HP-guar, or combinations thereof. Other compounds may also be added to the formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers. Combinations and mixtures of the above agents are also suitable. According to some embodiments, the concentration of viscosity enhancing agent or combination of agents ranges from about 0.2% to about 10% w/v, or any specific value within said range.

In one a preferred embodiment of the invention, the viscosity enhancing component comprises hydroxypropyl methylcellulose (Hypromellose or HPMC). According to some embodiments, the concentration of HPMC ranges from about 0.5% to about 2% w/v, or any specific value within said range. According to some embodiments, the concentration of HPMC ranges from about 0.5% to about 1.5% w/v, or any specific value within said range. According to some embodiments, the concentration of HPMC ranges from about 0.5% to about 1% w/v, or any specific value within said range. According to some embodiments, the concentration of HPMC ranges from about 0.6% to about 1% w/v, or any specific value within said range. In a preferred embodiment, the concentration of HPMC ranges from about 0.7% to about 0.9% w/v, or any specific value within said range (i.e., about 0.70%, about 0.71%, about 0.72%, about 0.73%, about 0.74%, about 0.75%, about 0.76%, about 0.77%, about 0.78%, about 0.79%, about 0.80%, about 0.81%, about 0.82%, about 0.83%, about 0.84%, about 0.85%, about 0.86%, about 0.87%, about 0.88%, about 0.89%, or about 0.90%).

In another preferred embodiment, the viscosity enhancing component comprises carboxymethyl cellulose sodium.

### Tonicity Adjusting Agents

The aqueous ophthalmic formulations/compositions of the present invention further comprise a tonicity agent or combination of tonicity agents. According to some embodiments, the ophthalmic formulations/compositions may include an effective amount of a tonicity adjusting component. Among the suitable tonicity adjusting components that can be used are those conventionally used in contact lens care products such as various inorganic salts. Polyols and polysaccharides can also be used to adjust tonicity. The amount of tonicity adjusting component is effective to provide an osmolality from 200 mOsmol/kg to 400 mOsmol/kg or from 260 mOsmol/kg to 350 mOsmol/kg.

Preferably, the tonicity component comprises a physiologically balanced salt solution that mimics the mineral composition of tears. According to some embodiments, tonicity may adjusted by tonicity enhancing agents that include, for example, agents that are of the ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄ or boric acid. Non-ionic tonicity enhancing agents are, for example, urea, glycerol, sorbitol, mannitol, propylene glycol, or dextrose.

According to some embodiments, the tonicity component comprises two or more of NaCl, KCl, ZnCl₂, CaCl₂, and MgCl₂ in a ratio that provides an osmolality range of about 140 to about 400 mOsm/kg, preferably about 240 to about 330 mOsm/kg, preferably about 260 to about 300 mOsm/kg, with the most preferred osmolality of approximately 270 mOsm/kg. According to some embodiments, the tonicity component comprises three or more of NaCl, KCl, ZnCl₂, CaCl₂, and MgCl₂ in a ratio that provides an osmolality range of about 140 to about 400 mOsm/kg, preferably about 240 to about 330 mOsm/kg, preferably about 260 to about 300 mOsm/kg, with the most preferred osmolality of approximately 270 mOsm/kg. According to some embodiments, the tonicity component comprises four or more of NaCl, KCl, ZnCl₂, CaCl₂, and MgCl₂ in a ratio that provides an osmolality range of about 140 to about 400 mOsm/kg, preferably about 240 to about 330 mOsm/kg, preferably about 260 to about 300 mOsm/kg, with the most preferred osmolality of approximately 270 mOsm/kg. According to some embodiments, the tonicity component comprises NaCl, KCl, ZnCl₂, CaCl₂, and MgCl₂ in a ratio that provides an osmolality range of about 140 to about 400 mOsm/kg, preferably about 240 to about 330 mOsm/kg, preferably about 260 to about 300 mOsm/kg, with the most preferred osmolality of approximately 270 mOsm/kg.

According to some embodiments, NaCl ranges from about 0.1 to about 1% w/v, preferably from about 0.2 to about 0.8% w/v, more preferably about 0.39% w/v. According to some embodiments, KCl ranges from about 0.02 to about 0.5% w/v, preferably about 0.05 to about 0.3% w/v, more preferably about 0.14% w/v. According to some embodiments, CaCl₂ ranges from about 0.0005 to about 0.1% w/v, preferably about 0.005 to about 0.08% w/v, more preferably about 0.06% w/v. According to some embodiments, MgCl₂ ranges from about 0.0005 to about 0.1% w/v, preferably about 0.005 to about 0.08% w/v, more preferably about 0.06% W/V. According to some embodiments, ZnCl₂ ranges from about 0.0005 to about 0.1% w/v, preferably about 0.005 to about 0.08% w/v, more preferably about 0.06% W/V.

According to some embodiments, the ophthalmic formulations of the present invention may be adjusted with tonicity agents to approximate the osmotic pressure of normal lachrymal fluids (isotonic) which is equivalent to a 0.9% solution of sodium chloride or a 2.5% solution of glycerol. An osmolality of about 225 to 400 mOsm/kg is preferred, more preferably 280 to 320 mOsm.

### Buffers and pH

The ophthalmic formulations/compositions of the present invention also include a buffer system. As used in this application, the terms “buffer” or “buffer system” is meant a compound that, usually in combination with at least one other compound, provides a buffering system in solution that exhibits buffering capacity, that is, the capacity to neutralize, within limits, either acids or bases (alkali) with relatively little or no change in the original pH. According to some embodiments, the buffering components are present from 0.05% to 2.5% (w/v) or from 0.1% to 1.5% (w/v).

Preferred buffers include borate buffers, phosphate buffers, citrate buffers, and combinations and mixtures thereof. Borate buffers include, for example, boric acid and its salts, for example, sodium borate or potassium borate. Borate buffers also include compounds such as potassium tetraborate or potassium metaborate that produce borate acid or its salt in solutions. Citrate buffer system may be comprised of citric acid and/or sodium citrate

A phosphate buffer system preferably includes one or more monobasic phosphates, dibasic phosphates and the like. Particularly useful phosphate buffers are those selected from phosphate salts of alkali and/or alkaline earth metals. Examples of suitable phosphate buffers include one or more of sodium dibasic phosphate (Na₂HPO₄), sodium monobasic phosphate (NaH₂PO₄) and potassium monobasic phosphate (KH₂PO₄). The phosphate buffer components frequently are used in amounts from 0.01% or to 0.5% (w/v), calculated as phosphate ion.

A preferred buffer system is based upon boric acid/borate, a mono and/or dibasic phosphate salt/phosphoric acid or a combined boric/phosphate buffer system. For example a combined boric/phosphate buffer system can be formulated from a mixture of sodium borate and phosphoric acid, or the combination of sodium borate and the monobasic phosphate.

In a combined boric/phosphate buffer system, the solution comprises about 0.05 to 2.5% (w/v) of a phosphoric acid or its salt and 0.1 to 5.0% (w/v) of boric acid or its salt. The phosphate buffer is used (in total) at a concentration of 0.004 to 0.2 M (Molar), preferably 0.04 to 0.1 M. The borate buffer (in total) is used at a concentration of 0.02 to 0.8 M, preferably 0.07 to 0.2 M. Additionally, 2.5% (w/v) of a citric acid or its salt may be added to this buffer system.

In a combined boric/citrate buffer system, the solution comprises about 0.05 to 2.5% (w/v) of a citric acid or its salt and 0.1 to 5.0% (w/v) of boric acid or its salt. The citrate buffer is used (in total) at a concentration of 0.004 to 0.2 M (Molar), preferably 0.04 to 0.1 M. The borate buffer (in total) is used at a concentration of 0.02 to 0.8 M, preferably 0.07 to 0.2 M. Additionally, 2.5% (w/v) of a phosphoric acid or its salt may be added to this buffer system.

Other known buffer compounds can optionally be added to the lens care compositions, for example, citrates, sodium bicarbonate, TRIS, and the like. Other ingredients in the solution, while having other functions, may also affect the buffer capacity. For example, EDTA, often used as a complexing agent, can have a noticeable effect on the buffer capacity of a solution.

According to some embodiments, the pH of the aqueous ophthalmic solution is at or near physiological pH. Preferably, the pH of the aqueous ophthalmic solution is between about 6.8 to about 7.7 (e.g., about 6.8, about 6.9, about 7.0, about 7.1, about 7.2, about 7.3, about 7.4, about 7.5, about 7.6, or about 7.7). According of some embodiments, the pH of the aqueous ophthalmic solution is between about 6.8 to about 7.5. According of some embodiments, the pH of the aqueous ophthalmic solution is between about 6.8 to about 7.4. According of some embodiments, the pH of the aqueous ophthalmic solution is between about 7.0 to about 7.4. According of some embodiments, the pH of the aqueous ophthalmic solution is between about 7.2 to about 7.4. According of some embodiments, the pH of the aqueous ophthalmic solution is between about 7.2 to about 7.5. According to some embodiments, the pH is adjusted with base (e.g., 1N sodium hydroxide) or acid (e.g., 1N hydrochloric acid).

### Viscosity

According to some embodiments, the ophthalmic formulations of the present invention has a viscosity that ranges from about 30 to about 150 centipoise (cpi), preferably about 30 to about 130 cpi, more preferably about 50 to about 120 cpi, more preferably about 60 to about 115 cpi, even more preferably about 60-90 cpi (or any specific value within said ranges). According to preferred embodiments, the ophthalmic formulations of the present invention has a viscosity that ranges from about 70 to about 88 cpi, or any specific value within said range (i.e., about 70 cpi, about 71 cpi, about 72 cpi, about 73 cpi, about 74 cpi, about 75 cpi, about 76 cpi, about 77 cpi, about 78 cpi, about 79 cpi, about 80 cpi, about 81 cpi, about 82 cpi, about 83 cpi, about 84 cpi, about 85 cpi, about 86 cpi, about 87 cpi, or about 88 cpi).

The viscosity of the ophthalmic formulations of the invention may be measured according to standard methods known in the art, such as use of a viscometer or rheometer. One of ordinary skill in the art will recognize that factors such as temperature and shear rate may effect viscosity measurement. In a particular embodiment, viscosity of the ophthalmic formulations of the invention is measured at 20° C.+/−1° C. using a Brookfield Cone and Plate Viscometer Model VDV-III Ultra⁺ with a CP40 or equivalent Spindle with a shear rate of approximately 22.50+/−approximately 10 (1/sec), or a Brookfield Viscometer Model LVDV-E with a SC4-18 or equivalent Spindle with a shear rate of approximately 26+/−approximately 10 (1/sec)). Alternatively, viscosity of the ophthalmic formulations of the invention is measured at 25° C.+/−1° C. In preferred embodiments, HPMC is slowly added to achieve 0.75% a viscosity of from about 70 to about 88 cpi at 25° C.+/−1° C.

### Preferred Ophthalmic Formulations

In one particular embodiment, the ophthalmic formulation of the invention is a 0.8% hypromellose solution comprised of the following: 8.0 mg/ml Hypromellose, USP Type 2910 (Methocel® E4M Premium—Hydroxypropyl methylcellulose); 8.0 mg/ml sodium chloride, USP; 0.15 mg/ml edetate sodium, USP; Sodium hydroxide, 5 N or Hydrochloric Acid, 5 N to adjust pH to 7.4; and purified water, USP (QS to 1 ml). Optionally, this 0.8% hypromellose solution contains a preservative such as benzalkonium chloride or a derivative thereof (e.g., Polyquad®), a stabilized oxychloro complex (e.g., Purite®), sodium perborate, or sorbate. Preferably, this 0.8% hypromellose solution is formulated at a viscosity of approximately 70-88 cpi.

In another particular embodiment, the ophthalmic formulation of the invention is a 0.8% hypromellose solution formulated for unit dose comprised of the following: 8.0 mg/ml Hypromellose, USP Type 2910 (Methocel® E4M Premium—Hydroxypropyl methylcellulose); 10 mg/ml glycerin, USP; 4.8 mg/ml sodium chloride, USP; 0.8 mg/ml potassium chloride, USP; 0.08 mg/ml calcium chloride, USP; 0.05 mg/ml magnesium chloride, USP; 1.7 mg/ml sodium citrate, USP; NaOH/HCl to adjust pH to 7.4; and purified water, USP (QS to 1 ml). Optionally, this 0.8% hypromellose solution contains a preservative such as benzalkonium chloride or a derivative thereof (e.g., Polyquad®), a stabilized oxychloro complex (e.g., Purite®), sodium perborate, or sorbate. Preferably, this 0.8% hypromellose unit dose solution is formulated at a viscosity of 70-88 cpi.

In still another particular embodiment, the ophthalmic formulation of the invention is a 0.8% hypromellose solution formulated for multi-dose comprised of the following: 8.0 mg/ml Hypromellose, USP Type 2910 (Methocel® E4M Premium—Hydroxypropyl methylcellulose); 10 mg/ml glycerin, USP; 4.8 mg/ml sodium chloride, USP; 0.8 mg/ml potassium chloride, USP; 0.08 mg/ml calcium chloride, USP; 0.05 mg/ml magnesium chloride, USP; 5.0 mg/ml boric acid, NF; 1.0 mg/ml sorbic acid, NF; 1.0 mg/ml disodium edetate, USP; NaOH/HCl to adjust pH to 7.4; and purified water, USP (QS to 1 ml). Optionally, this 0.8% hypromellose solution contains a preservative such as benzalkonium chloride or a derivative thereof (e.g., Polyquad®), a stabilized oxychloro complex (e.g., Purite®), sodium perborate, or sorbate. Preferably, this 0.8% hypromellose multi-dose solution is formulated at a viscosity of 70-88 cpi.

### Pharmaceutical Compositions

The aqueous ophthalmic formulations of the present invention are suitable for use as artificial tear products to relieve symptoms of dry eye. Alternatively, the formulations of the present invention may act as a vehicle for an ophthalmic drug. Ophthalmic drugs suitable for use in the formulations of the present invention include, but are not limited to: anti-glaucoma agents, such as beta-blockers including timolol, betaxolol, levobetaxolol, carteolol, miotics including pilocarpine, carbonic anhydrase inhibitors, prostaglandins, seretonergics, muscarinics, dopaminergic agonists, adrenergic agonists including apraclonidine and brimonidine; anti-angiogenesis agents; anti-infective agents including quinolones such as ciprofloxacin, and aminoglycosides such as tobramycin and gentamicin; steroidal anti-inflammatory agents such as rimexolone, tetrahydrocortisol, fluticasone, beclomethasone, budesonide, triamcinolone, and mometasone; non-steroidal anti-inflammatory agents (NSAIDs) such as naproxen, flurbiprofen, oxaprozin, ibuprofen, ketoprofen, fenoprofen; ketorolac tromethamine, sulindac, indomethacin, etodolac, diclofenac, bromfenac, suprofen, nepafenac, amfenac, aspirin, salsalate, diflunisal, choline magnesium trisalicylate (CMT), acetaminophen, nabumetone, piroxicam, meloxicam, mefenamic acid, meclofenamate, flufenamic acid, tolmetin, phenylbutazone, elecoxib, valdecoxib, parecoxib, etoricoxib, and luaricoxib; growth factors, such as EGF; immunosuppressant agents; anti-allergic or antihistamine agents such as astemizole, azelastine, antazoline, brompheniramine, chlorpheniramine, cetirizine, clemastine, desloratidine, dexbrompheniramine, diphenhydramine, doxylamine, ebastine, emedastine, epinastine, fexofenadine, hydroxyzine, ketotifen, levocabastine, levocetirizine, loratidine, mequitazine, mizolastine, norketotifen, olopatadine, oxatomide, phenindamine, pheniramine, pyrilamine, terfenidine, and triprolidine; vasoconstrictors such as naphazoline, antolazine, oxymetazoline, phenylephrine, and tetrahydrozoline; or any combination thereof. Any of the above-referenced ophthalmic drugs may be present in the form of a pharmaceutically acceptable salt including all esters and pharmaceutically acceptable salts thereof. Compositions of the present invention may also include combinations of the above-referenced ophthalmic drugs, such as combinations of (i) a beta-blocker selected from the group consisting of betaxolol and timolol, and (ii) a prostaglandin selected from the group consisting of latanoprost; 15-keto latanoprost; travoprost; and unoprostone isopropyl. Alternatively, the pharmaceutical compositions of the invention may be administered in conjunction (simultaneously or sequentially) with pharmaceutical compositions comprising other active ingredients, including, but not limited to, vasoconstrictors, anti-allergenic agents, anti-infectives, steroids, anesthetics, anti-inflammatories, analgesics, dry eye agents (e.g. secretagogues, mucomimetics, polymers, lipids, antioxidants), etc.

Although the amount of an ophthalmic drug included in the compositions of the present invention will be whatever amount is therapeutically effective and will depend upon a number of factors, including the identity and potency of the chosen drug. The therapeutically effective amount of an ophthalmic drug in the formulation will depend on absorption, inactivation, and excretion rates of the drug as well as the delivery rate of the compound from the formulation, and will be suitable for short or long term use for the treatment of acute or chronic conditions, respectively. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any ophthalmic drug may also vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment. The effectiveness of any formulation and method of treatment or prevention may be assessed by administering the formulation and assessing the effect of the administration by measuring one or more indices associated with the efficacy of the ophthalmic drug composition and with the degree of comfort to the patient, as described herein, and comparing the post-treatment values of these indices to the values of the same indices prior to treatment or by comparing the post-treatment values of these indices to the values of the same indices using a different formulation.

According to some embodiments, the total concentration of drug will generally be about 5% or less. For example, the tear substitute compositions of the invention may be used in combination with another pharmaceutical composition, such as a prescription drug like Restasis™ (cyclosporine ophthalmic emulsion, 0.05%). It may be used simultaneously with another pharmaceutical composition, or in sequence. For example, the tear substitute compositions of the invention may be administered to a subject in the ramp up period before another administered pharmaceutical begins to be effective in the subject. In certain embodiments, the tear substitute compositions of the invention may be used in a manner such that they serve as a replacement for a prescription drug like Restasis™.

The active ingredients of the pharmaceutical compositions may be in the form of a pharmaceutically acceptable salt.

Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions, or those appropriate for sustained release. Accordingly to some embodiments, the pharmaceutical compositions according to the present invention will be formulated as solutions, suspensions, ointments, gels, sustained release formulation, and other dosage forms for topical administration or for sustained release delivery.

Any of a variety of carriers may be used in the formulations of the present invention including water, mixtures of water and water-miscible solvents, such as C₁- to C₇-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient.

### Preservative

Preferably the formulations of the present invention are preservative free. Such formulations would be useful for patients with dry eye, patients who wear contact lenses, or those who use several topical ophthalmic drops and/or those with an already compromised ocular surface (e.g. dry eye) wherein limiting exposure to a preservative may be more desirable.

In certain embodiments, however, the formulations of the invention may additionally comprise a preservative. A preservative may typically be selected from a quaternary ammonium compound such as benzalkonium chloride, benzoxonium chloride or the like. Benzalkonium chloride is better described as: N-benzyl-N—(C8-C18 alkyl)-N,Ndimethylammonium chloride. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II, sorbic acid, and stabilized oxychloro complexes (e.g., Purite®). Preferred preservatives are quaternary ammonium compounds, in particular benzalkonium chloride or its derivative such as Polyquad (see U.S. Pat. No. 4,407,791), alkyl-mercury salts, parabens and stabilized oxychloro complexes (e.g., Purite®). Where appropriate, a sufficient amount of preservative is added to the ophthalmic composition to ensure protection against secondary contaminations during use caused by bacteria and fungi.

### Solubilizer

The formulation of the invention may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

### Excipients

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

### Packaging

The formulations of the present invention may be packaged as either a single dose product (i.e., unit dose) or a multi-dose product. The single dose product is sterile prior to opening of the package and all of the composition in the package is intended to be consumed in one or several applications to one or both eyes of a patient. The use of an antimicrobial preservative to maintain the sterility of the composition after the package is opened is generally unnecessary. The formulations, if an ointment formulation, may be packaged as appropriate for an ointment, as is known to one of skill in the art.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and other publications by the Food and Drug Administration, and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” or “AET” requirements. (The acronym “PET” stands for “preservative efficacy testing”; the acronym “AET” stands for “anti-microbial efficacy testing”).

The use of a single dose (i.e., unit dose) packaging arrangement eliminates the need for an anti-microbial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation, or patients using multiple preserved products. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

### Methods of Use

As described herein, the ophthalmic formulations/compositions of the present invention find particular utility as a treatment for the signs and symptoms of dry eye disease. Clinical signs of dry eye disease include but are not limited to increased corneal staining, reduced tear film integrity as evidenced by a reduced or shortened tear film break up time or low ocular protection index (OPI). Symptoms of dry eye disease include but are not limited to ocular discomfort. In particular, the ophthalmic formulations/compositions of the present invention are useful for reducing corneal staining, improving tear film integrity (i.e., increasing tear film break up time (TFBUT) and the ocular protection index) and reducing ocular discomfort levels in subjects having dry eye disease. The ophthalmic formulations/compositions of the present invention find further utility as a moisturizing and lubricating eye drop (i.e., an artificial tear solution), a delivery vehicle for ophthalmic drugs, and as a contact lens wetting and lubricating solution.

Provided also are methods of increasing the tear film break-up time (TFBUT) of a subject's tear film, comprising administering to the eye surface of the subject in need thereof an ophthalmic formulations/compositions of the present invention.

Provided also are methods of increasing the ocular protection index (OPI) of a subject's eye, comprising administering to the eye surface of the subject in need thereof an ophthalmic formulations/compositions of the present invention.

Provided also are methods for improving, treating, relieving, inhibiting, preventing, or otherwise decreasing ocular discomfort in a subject comprising administering to the eye surface of the subject in need thereof an ophthalmic formulations/compositions of the present invention.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determine the optimum time and/or amount of administration, which will require no more than routine experimentation consisting of monitoring the subject and adjusting the dosage and/or timing.

The combined use of several ophthalmic drugs formulated into the compositions of the present invention may reduce the required dosage for any individual component because the onset and duration of effect of the different components may be complimentary. In such combined therapy, the different ophthalmic drugs may be delivered together or separately, and simultaneously or at different times within the day.

Efficacy of the formulations and compositions of the invention in treating and preventing the signs and symptoms associated with dry eye disease and/or ocular irritation may be assessed by measuring changes in tear film break-up time, changes in ocular protection index, and level of ocular comfort. An increase in tear film break-up time and/or ocular protection index in a subject, following administration of the formulations and compositions of the invention as compared to TFBUT and or OPI prior to administration, indicates that the formulation is effective in treating and preventing signs and symptoms associated with dry eye disease and/or ocular irritation.

The ophthalmic formulations of the present invention effectively enhance tear film stability. One measure of tear film stability is an increase in tear film break up time (TFBUT) when measured post-instillation of the ophthalmic formulation into the eye as compared to tear film break up time measured prior to instillation of the ophthalmic formulation into the eye (i.e., baseline TFBUT). For example, without limitation, tear film break up time is increased whereby the TFBUT is about 2-fold greater, about 3-fold greater, about 4-fold greater, about 5-fold greater, or about six-fold greater or more, when measured post instillation of the ophthalmic formulation into the eye as compared to tear film break up time measured prior to instillation of the ophthalmic formulation into the eye (i.e., baseline TFBUT).

One method of determining a clinically meaningful increase in TFBUT is an increase in Ocular Protection Index (OPI) when measured post-instillation of the ophthalmic formulation into the eye as compared to OPI measured prior to instillation of the ophthalmic formulation into the eye (i.e., baseline OPI). For example, without limitation, the ocular protection index is increased whereby the OPI is about 2-fold greater, about 3-fold greater, about 4-fold greater, about 5-fold greater, or about 6-fold greater or more, when measured post-instillation of the ophthalmic formulation into the eye as compared to OPI measured prior to instillation of the ophthalmic formulation into the eye (i.e., baseline OPI). Ocular irritation/discomfort is effectively decreased whereby patient assessment of ocular discomfort is less when measure post-instillation of the ophthalmic formulation into the eye as compared to ocular discomfort measured prior to instillation of the ophthalmic formulation into the eye.

TFBUT may be measured using various methods, including but not limited to illumination of the eye following instillation of sodium fluorescein in the eye, or equivalents thereof. An increase in ocular comfort or decrease in ocular discomfort in a subject following administration of the formulations and compositions of the invention as compared to ocular comfort level prior to administration, indicates that the formulation is effective in treating and preventing signs and symptoms associated with dry eye disease and/or ocular irritation. Ocular comfort level may be assessed by various methods, including but not limited to subjective scales (for example but not limited to, standardized subjective scales that determine ocular discomfort as mild, moderate, sever, or 0, 1, 2, 3, 4, etc., or other appropriate scale), reflexive response (e.g., flinch-reflex), and physiological response, including but not limited to changes in heart rate, blood pressure, and perspiration levels.

### Kits

In still another embodiment, this invention provides kits for the packaging and/or storage and/or use of the formulations described herein, as well as kits for the practice of the methods described herein. Thus, for example, kits may comprise one or more containers containing one or more ophthalmic solutions, ointments, gels, sustained release formulations or devices, suspensions or formulations, tablets, or capsules of this invention. The kits can be designed to facilitate one or more aspects of shipping, use, and storage.

The kits may optionally include instructional materials containing directions (i.e., protocols) disclosing means of use of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

All publications and patents mentioned herein are hereby incorporated by reference in their entirety as if each individual publication or patent was specifically and individually indicated to be incorporated by reference. In case of conflict, the present application, including any definitions herein, will control.

### Methods of Manufacture

In accordance with the present invention, there is also provided a method of manufacture of a comfortable ophthalmic formulation for instillation into the eye. Preferably, the ophthalmic formulation comprises one or more tear substitute components such as those described herein.

According to preferred embodiments, there is provided a method of manufacture of an ophthalmic formulation for instillation into the eye comprising: heating a solution base to at least 80° C.; mixing the aqueous solution at an appropriate rate, depending on the tank and impellar design, which prevents clumping; adding a viscosity agent at a rate that avoids clumping of the viscosity agent to achieve a target viscosity; and adding tonicity agent(s), buffer(s) and optional ophthalmic drug. According to some embodiments, the target viscosity is from about 70 to 88 cpi. In preferred embodiments, the viscosity agent is HPMC, which is added to a concentration of 0.72% to 0.80% to achieve a viscosity of 70 to 88 cpi.

According to preferred embodiments the ophthalmic formulation is prepared in commercial scale batches. Commercial scale batches include batches from about 50 liters to about 500 liters or anywhere between (e.g., 220 liters). Preferred embodiments include batches from about 50 liters to about 500 liters; batches from about 50 liters to about 400 liters; batches from about 50 liters to about 300 liters; batches from about 50 liters to about 250 liters; batches from about 50 liters to about 220 liters; batches from about 50 liters to about 200 liters; batches from about 50 liters to about 100 liters; batches from about 100 liters to about 220 liters; batches from about 100 liters to about 200 liters; batches from about 120 liters to about 200 liters; batches from about 150 liters to about 220 liters; batches from about 150 liters to about 200 liters; or batches from about 180 liters to about 220 liters.

The solution base is added to the mixing vessel or kettle tank (e.g., 220 L kettle tank). Preferably the solution base is sterile water. The solution base is heated to below the boiling point of water. Preferably the solution base is heated to at least 99° C.; at least 98° C.; at least 97° C.; at least 96° C.; at least 95° C.; at least 94° C.; at least 93° C.; at least 92° C.; at least 91° C.; at least 90° C.; at least 89° C.; at least 88° C.; at least 87° C.; at least 86° C.; or at least 85° C. According to some embodiments, the solution base is heated to from about 80° C. to about 95° C.; from about 85° C. to about 95° C.; from about 90° C. to about 95° C.; from about 80° C. to about 90° C.; or from about 85° C. to about 95° C.

In accordance with the present invention, the use of an optimized rate of mixing in the mixing vessel increases solubilization of the viscosity agent and prevents clumping. The limit on the low end of the movement rate is dependent on the controlled rate for adding the viscosity agent. Thus, very slow movement will eventually result in complete mixing. According to preferred embodiments, a high rate of mixing is used to increase solubilization of the viscosity agent and prevent clumping. The mechanism for mixing can be any mechanism known in the art. Preferably, the mixing of stirring is achieved using a high shear mixing blade, low shear mixing blade, vortex, or side-scraper. A side-scraper is preferred.

Preferred rates of movement that will ensure mixing will depend on the tank and impellar design. In some embodiments, the preferred rate of movements that will ensure mixing within a time useful for most purposes are from 5 to 5,000 rotations per minute (rpm), more preferably 5 to 500 rpm, and most preferably about 5 to 100 rpm. According to some embodiments, the rate of movement of a mixing blade is from about 10 to about 100 rpm; from about 10 to about 90 rpm; from about 10 to about 80 rpm; from about 10 to about 70 rpm; from about 10 to about 60 rpm; from about 10 to about 50 rpm; from about 10 to about 40 rpm; from about 10 to about 30 rpm; from about 10 to about 25 rpm; from about 10 to about 20 rpm; or from about 5 to about 10 rpm.

In accordance with the present invention, the viscosity agent is added at a controlled rate in order to avoid clumping and increase solubilization. According to preferred embodiments, the rate of addition of the viscosity agent is from about 1 to about 1000 grams per minute (gm/min). According to some embodiments, the rate of addition of the viscosity agent is from about 5 to about 1000 (gm/min); from about 10 to about 1000 (gm/min); from about 50 to about 1000 (gm/min); from about 100 to about 1000 (gm/min); from about 200 to about 1000 (gm/min); from about 300 to about 1000 (gm/min); from about 400 to about 1000 (gm/min); from about 500 to about 1000 (gm/min); from about 600 to about 1000 (gm/min); from about 700 to about 1000 (gm/min); from about 800 to about 1000 (gm/min); from about 900 to about 1000 (gm/min); from about 5 to about 900 (gm/min); from about 5 to about 750 (gm/min); from about 5 to about 500 (gm/min); from about 5 to about 250 (gm/min); from about 5 to about 100 (gm/min); from about 5 to about 90 (gm/min); from about 5 to about 80 (gm/min); from about 5 to about 70 (gm/min); from about 5 to about 60 (gm/min); from about 5 to about 50 (gm/min); from about 5 to about 40 (gm/min); from about 5 to about 30 (gm/min); or from about 5 to about 20 (gm/min) from about 5 to about 10 (gm/min).

A relationship exists between the rate of mixing and the rate of addition of the viscosity agent to achieve solubilization and avoid clumping. For example, the viscosity agent may be added to the solution base at a rate of approximately 10 grams per minute (gm/min) while stirring with a low shear mixing blade or side scraper blade at 10 to 100 rotations per minute (rpm). The rate of addition of the viscosity agent and rate of mixing may form a relationship such as that provided in Table 1 as follows.

TABLE 1
Relationship between rate of addition
of viscosity agent and rate of mixing

| grams per minute (gm/min) | rpm |
|---|---|
| 10 | 100 |
| 9 | 90 |
| 8 | 80 |
| 7 | 70 |
| 6 | 50 |
| 4 | 40 |
| 3 | 30 |
| 2 | 20 |
| 1 | 10 |
| 0.5 | 5 |

The relationship described in Table 1 above may be adjusted to optimize the solubilization and avoid clumping of the viscosity agent. For example, the relationship described in Table 1 above may be adjusted by increasing the rate of addition of the viscosity agent by 2×, 3×, 4×, 5×, 6×, 7×, 8×, 9×, 10×, or greater (e.g., 20×, 30×, 40×, 50×, 60×, 70×, 80×, 90×, or 100×). Thus, a 20× increase in the rate of addition of the viscosity agent would form a relationship with the mixing speed as shown in Table 2 below.

TABLE 2
20× adjusted relationship between rate
of addition of viscosity agent and rate of mixing

| grams per minute (gm/min) | rpm |
|---|---|
| 200 | 100 |
| 180 | 90 |
| 160 | 80 |
| 140 | 70 |
| 120 | 50 |
| 100 | 40 |
| 80 | 30 |
| 60 | 20 |
| 40 | 10 |
| 10 | 5 |

The relationship shown in Table 3 is also appropriate.

TABLE 3
Alternative 20× adjusted relationship between
rate of addition of viscosity agent and rate of mixing

| grams per minute (gm/min) | rpm |
|---|---|
| 200 | 1000 |
| 180 | 900 |
| 160 | 800 |
| 140 | 700 |
| 120 | 500 |
| 100 | 400 |
| 80 | 300 |
| 60 | 200 |
| 40 | 100 |
| 10 | 50 |

The viscosity agent is added until the target viscosity is achieved, as described herein elsewhere. Once the viscosity agent (e.g., HPMC) has been wetted and dispersed, the buffer and salts may be added. The solution may be bulk sterilized, cooled, and pH adjusted to the desired level. The resulting formulation may then be packaged as desired.

### DEFINITIONS

For convenience, before further description of the present invention, certain terms employed in the specification, examples, and appended claims are collected here. These definitions should be read in light of the remainder of the disclosure and understood as by a person of skill in the art.

The term “acute” as used herein denotes a condition having a rapid onset, and symptoms that are severe but short in duration.

The term “analgesic” as used herein denotes a compound/formulation for the management of intermittent and/or chronic physical discomfort, suitable for long term use.

The term “anesthetic” or “anesthesia” as used herein denotes a compound/formulation for the management of acute physical pain, suitable for short term, temporary use, which has an effect that produces numbing or decreased sensitivity in the body part/organ to which the compound/formulation is administered (e.g., decreased corneal sensitivity of the eye).

The term “aqueous” typically denotes an aqueous composition wherein the carrier is to an extent of >50%, more preferably >75% and in particular >90% by weight water.

The term “chronic” as defined herein is meant a persistent, lasting condition, or one marked by frequent recurrence, preferably a condition that persists/recurs for greater than 3 months, more preferably greater than 6 months, more preferably greater than 12 months, and even more preferably greater than 24 months.

The term “comfortable” as used herein refers to a sensation of physical well being or relief, in contrast to the physical sensation of pain, burning, stinging, itching, irritation, or other symptoms associated with physical discomfort.

The term “comfortable ophthalmic formulation” as used herein refers to an ophthalmic formulation which provides physical relief from symptoms associated with dry eye disease and/or ocular discomfort, and only causes an acceptable level of pain, burning, stinging, itching, irritation, or other symptoms associated with ocular discomfort, when instilled in the eye, which are less than those seen with dosing with current concentrations on the market.

The term “dry eye” as used herein, refers to inadequate tear production and/or abnormal tear composition. Causes of dry eye disease as defined herein include but are not limited to the following: idiopathic, congenital alacrima, xerophthalmia, lacrimal gland ablation, and sensory denervation; collagen vascular diseases, including rheumatoid arthritis, Wegener's granulomatosis, and systemic lupus erythematosus; Sjögren's syndrome and autoimmune diseases associated with Sjögren's syndrome; abnormalities of the lipid tear layer caused by blepharitis or rosacea; abnormalities of the mucin tear layer caused by vitamin A deficiency; trachoma, diphtheric keratoconjunctivitis; mucocutaneous disorders; aging; menopause; and diabetes. Dry eye signs and/or symptoms as defined herein may also be provoked by other circumstances, including but not limited to the following: prolonged visual tasking; working on a computer; being in a dry environment; ocular irritation; contact lenses, LASIK and other refractive surgeries; fatigue; and medications such as isotretinoin, sedatives, diuretics, tricyclic antidepressants, antihypertensives, oral contraceptives, antihistamines, nasal decongestants, beta-blockers, phenothiazines, atropine, and pain relieving opiates such as morphine.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a sign and/or symptom of dry eye and/or eye irritation, or prevent or treat dry eye and/or eye irritation. The effective amount may vary depending on such factors as the disease or condition being treated, the particular composition being administered, or the severity of the disease or condition. One of skill in the art may empirically determine the effective amount of a particular agent without necessitating undue experimentation.

As used herein, the term “NSAID” means an opthalmologically acceptable nonsteroidal anti-inflammatory drug or a pharmaceutically acceptable salt thereof. The term “low dose NSAID” means an amount of an opthalmologically acceptable nonsteroidal anti-inflammatory drug or a pharmaceutically acceptable salt thereof, which reduces ocular discomfort without producing anesthesia, and which would be expected to have reduced adverse effects associated with current FDA approved formulations of NSAIDs marketed for the treatment of acute ocular inflammation and pain, including without limitation, corneal damage, delayed wound healing, and ocular discomfort.

A “patient,” “subject,” or “host” to be treated by the subject method refers to either a human or non-human animal, such as a primate, mammal, and vertebrate

The phrase “pharmaceutically acceptable” is art-recognized and refers to compositions, polymers and other materials and/or salts thereof and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the surface of the eye. Each carrier must be “acceptable” in the sense of being compatible with the other ingredients of the composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, hydroxypropyl methylcellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) gums such as HP-guar; (22) polymers; and (23) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, ptoluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; tromethamine, and the like. See, e.g., J. Pharm. Sci., 66: 1-19 (1977).

The term “preventing,” when used in relation to a condition, such as dry eye and/or eye irritation, is art-recognized, and refers to administration of a composition which reduces the frequency of, or delays the onset of, signs and/or symptoms of a medical condition in a subject relative to a subject which does not receive the composition.

As used herein, the terms “tear substitute” and “artificial tear” may be used interchangeably, and each refers to one or more molecules or compositions, which lubricate, “wet,” approximate the consistency of endogenous tears, aid in natural tear build up, or otherwise provide temporary relief of dry eye signs and/or symptoms and conditions upon ocular administration, including without limitation a polymer (e.g., a cellulosic polymer), an ocular surface protectant, a demulcent, or other component found on the FDA monograph for tear substitutes. The term “tear substitute component” refers to one or more components thereof.

The term “treating” is an art-recognized term which refers to reducing or ameliorating at least one sign and/or symptom of any condition or disease.

### EXAMPLES

The invention now being generally described, it will be more readily understood by reference to the following examples which are included merely for purposes of illustration of certain aspects and embodiments of the present invention, and are not intended to limit the invention in any way.

### Example 1

### Manufacture of an Ophthalmic Formulation According to the Present Invention

This example provides a method of making an artificial tear solution that has the characteristics of 1) a high viscosity, providing increased artificial tear retention time on the ocular surface, and 2) hypo-tonicity, addressing the underlying hyper-tonic conditions characterized by dry eye, in a single physiologically-based, preservative-free eye-drop solution.

The Artificial Tear formulation consists of a demulcent/hydrogel (Hypromellose), a combination of salts, buffer, and water. The possible excipients and anticipated concentration ranges can be found in Table 4 below. The formulation is preservative free.

TABLE 4
Example of a Preservative Free Artificial
Tear Formulation of the Invention

| | | Anticipated |
|---|---|---|
| Component | Function | Concentration |
| Hypromellose, (HPMC | Viscosity enhancing | 0.8% target (0.70-0.90% |
| E4M Premium), USP | agent | possible range) |
| Sodium chloride, USP | tonicity agent | 0.08% |
| Edetate disodium, USP | Chelating agent | 0.015% |
| Sodium Hydroxide, 5N/ | pH adjusting agent | q.s. pH = 7.4 |
| Hydrochloric Acid, 5N | | |
| Sterile Water for | Solution base | q.s. |
| Injection, USP | | |

Generally, the difference in target concentration of HPMC is based on equipment, the rate of mixing differs between equipment used for large vs. small scale. The target concentration of HPMC at commercial scale is 0.75% target, while the target concentration of HPMC at lab bench scale is 0.8%.

In this example, the target scale is 0.75% HPMC (commercial scale). The manufacturing of the Artificial Tear utilizes a 220 L kettle tank with a side-scraper and a Blow/Fill/Seal (ASEP-TECH®) machine with a validated mold (e.g., 4-vial mold, however, the number of units can vary with the mold design). Water is added to the tank and subsequently heated with pressurized steam to a final temperature of 185 to 194° F. (90° C.).

HPMC is added to the kettle, which is stirred at optimal rate to avoid clumping as HPMC is added. While mixing, the HPMC is slowly added. The rate of addition is dependent on the mixing. Importantly, the mixing and rate of addition of HPMC is selected to avoid clumping of the HPMC on dispersion in the hot water. The initial process development for the Artificial Tear first involved identifying the appropriate compounding tank. It was initially expected that a compounding tank with vortex mixing would be sufficient for mixing the HPMC. However, after producing a few feasibility batches, it was apparent that the HPMC was not being fully recovered from the process due to inadequate mixing. Once the process was transferred to a kettle tank with a side-scraper, the HPMC was fully recovered from the process.

The end point of addition of HPMC is when the formula weight of HPMC has been added. Once the HPMC has been wetted and dispersed, the buffer and salts are added. Finally, the solution is bulk sterilized. The solution is cooled and the pH would be adjusted to 7.4.

The second phase of the process development involved optimizing the amount of HPMC to achieve the targeted viscosity range. In this example, the target viscosity was 70 to 88 cpi. The initial HPMC content used (0.80%), based on the phase 1/2 formulation, yielded a higher viscosity than target owing to the HPMC likely being better hydrated at the new manufacturing site. When the HPMC content was adjusted to 0.75%, the target viscosity was achieved and was able to be reproduced.

The third phase of development for this product will involve ensuring the viscosity target is met with the addition of the new salts and buffer(s) relative to the current HPMC, as well as the addition of an ophthalmic drug.

### Example 2

### Manufacture of an Ophthalmic Formulation According to the Present Invention

This example provides a method of making an artificial tear solution that has the characteristics of 1) a high viscosity, providing increased artificial tear retention time on the ocular surface, and 2) hypo-tonicity, addressing the underlying hyper-tonic conditions characterized by dry eye, in a single physiologically-based, preservative-free eye-drop solution.

The Artificial Tear formulation consists of a demulcent/hydrogel (Hypromellose), a combination of salts, buffer, and water. The possible excipients and anticipated concentration ranges can be found in Table 5 below. The formulation is preservative free.

| | | Anticipated |
|---|---|---|
| Component | Function | Concentration |
| Zinc chloride, USP | Tonicity agent | 0-0.80% |
| Magnesium chloride, USP | Tonicity agent | 0-0.80% |
| Potassium chloride, USP | Tonicity agent | 0-0.80% |
| Phosphate buffer | Buffer | 0-0.5% |
| Citrate buffer | Buffer | 0-0.5% |
| Borate buffer | Buffer | 0-0.5% |
| Sodium Hydroxide, 5N/ | pH adjusting agent | q.s. pH = 7.4 |
| Hydrochloric Acid, 5N | | |
| Sterile Water for | Solution base | q.s. |
| Injection, USP | | |

Generally, the difference in target concentration is based on equipment, the rate of mixing differs between equipment used for large vs. small scale. The target concentration of HPMC is 0.75% target on a commercial scale, and 0.8% target on a lab bench scale.

In this example, the target scale is 0.75% HPMC. The manufacturing of the Artificial Tear utilizes a 220 L kettle tank with a side-scraper and a Blow/Fill/Seal (ASEP-TECH®) machine with a validated mold (e.g., 4-vial mold, however the number of units can vary with mold design). Water is added to the tank and subsequently heated with pressurized steam to a final temperature of 185 to 194° F. (90° C.).

HPMC is added to the kettle, which is stirred at optimal rate to avoid clumping as HPMC is added. While mixing, the HPMC is slowly added. The rate of addition is dependent on the mixing. Importantly, the mixing and rate of addition of HPMC is selected to avoid clumping of the HPMC on dispersion in the hot water. The initial process development for the Artificial Tear first involved identifying the appropriate compounding tank. It was initially expected that a compounding tank with vortex mixing would be sufficient for mixing the HPMC. However, after producing a few feasibility batches, it was apparent that the HPMC was not being fully recovered from the process due to inadequate mixing. Once the process was transferred to a kettle tank with a side-scraper, the HPMC was fully recovered from the process.

The end point of addition of HPMC is when the formula weight of HPMC has been added. Once the HPMC has been wetted and dispersed, the buffer and salts are added. Finally, the solution is bulk sterilized. The solution is cooled and the pH would be adjusted to 7.4.

The second phase of the process development involved optimizing the amount of HPMC to achieve the targeted viscosity range. In this example, the target viscosity was 70 to 88 cpi. The initial HPMC content used (0.80%), based on the phase 1/2 formulation, yielded a higher viscosity than target owing to the HPMC likely being better hydrated at the new manufacturing site. When the HPMC content was adjusted to 0.75%, the target viscosity was achieved and was able to be reproduced.

The third phase of development for this product will involve ensuring the viscosity target is met with the addition of the new salts and buffer(s) relative to the current HPMC, as well as the addition of an ophthalmic drug.

### Example 3

### Assessment of the Effect of a 0.8% HMPC-Based Artificial Tear Solution on Clinical Signs of Dry Eye: Tear Film Break-Up Time (TFBUT)

The “tear film break-up time” or “TFBUT” test, an index of the severity of dry eye syndrome, can be used to measure the efficacy of a solution in maintaining the tear film. It is correlated with the degree of ocular discomfort a subject may feel. In a study involving hundreds of subjects, over 70% reported ocular discomfort within 1 second of tear film break-up. On average, the tear film in a normal eye breaks up in 7.1 seconds. In contrast, the tear film in a “dry eye” breaks up in an average of 3.2 seconds. Thus, agents having the ability to increase the TFBUT could be used in treating and preventing dry eye.

For example, the TFBUT may be assessed as follows. A patient's eye is first instilled with 2% sodium fluorescein. After the fluorescein instillation, the patient places his or her head in a slit lamp, and the investigator views the eye under cobalt blue illumination. The patient is instructed to blink three times and hold the eyes open at normal aperture after the third blink.

A stop watch is started when the eye is opened following the third blink, and is stopped when the investigator identifies a region of tear film break-up that has started to expand. The region of tear film break-up is identifiable by black voids in the otherwise confluent appearing tear film. The eye is video taped during the test.

A single-center, double-masked, randomized, vehicle-controlled study was performed to evaluate the safety and efficacy of a 0.8% HPMC ophthalmic solution (8.0 mg/ml Hypromellose, USP Type 2910 (Methocel® E4M Premium—Hydroxypropyl methylcellulose); 8.0 mg/ml sodium chloride, USP; 0.15 mg/ml edetate sodium, USP; Sodium hydroxide, 5 N or Hydrochloric Acid, 5 N to adjust pH to 7.4; and purified water, USP (QS to 1 ml)), designated herein as “AC-111” in treating the clinical symptoms of dry eye in subjects exposed to a Controlled Adverse Environment (CAE) chamber. In particular, the effect on tear-film break-up time (TFBUT) was evaluated. The CAE is a chamber in which humidity is controlled at a low level, and temperature, wind flow, lighting and visual tasking are all controlled. Patients who enter the CAE will develop ocular discomfort over time. This model allows for the precise evaluation of agents which can act to treat dry eye and/or ocular irritation.

Inclusion Criteria: Subject who participated in the study (N=30 for each treatment group) were required to meet each of the following criteria:

a) Are at least 18 years of age;
b) Are willing to provide written informed consent;
c) Are able and willing to follow instructions, including participation in study assessments, and can be present for the required study visits for the duration of the study;
d) Have a corrected visual acuity logMar +0.7 (ETDRS) or better in each eye;
e) Have a reported history of dry eye in each eye;
f) Use or have a desire to use an artificial tear substitute for dry eye symptoms in the past 6 months;
g) Have a ≧1 unit fluorescein staining score on the ORA scale in any region prior to exposure to the CAE in at least one eye;
h) Must report an ocular discomfort score of 3+ for two consecutive readings within 30 minutes of entering the CAE in both eyes. If subject enters the CAE with an ocular discomfort score of 3, then subject must record an ocular discomfort score of 4 for two consecutive readings within 30 minutes of CAE exposure in both eyes;
i) If female and of childbearing potential; agree to submit a urine sample and have a negative urine pregnancy test. Females are considered of childbearing potential unless they are surgically sterile, or post-menopausal.

Exclusion Criteria: Subjects who met the following criteria were excluded from the study:

a) Have contraindications to the use of the study medication (s);
b) Have known allergy or sensitivity to the study medication (s) or their components;
c) Have anterior blepharitis, which is deemed clinically significant and/or likely to interfere with study parameters in the opinion of the investigator;
d) Have any ocular infections (bacterial, viral or fungal) or active ocular inflammation (i.e., follicular conjunctivitis, iritis) or preauricular lymphadenopathy;
e) Report an ocular discomfort score of 4 in both eyes at time 0 of CAE exposure;
f) Use contact lenses within one week prior to the study visit;
g) Have any significant illness that could, in the opinion of the investigator, be expected to interfere with the trial parameters;
h) Be women who are currently pregnant, nursing, or planning a pregnancy; or women who have a positive pregnancy test;
i) Have participated in an investigational drug or device trial within 30 days of entering the study;
j) Are currently taking any topical ophthalmic prescription or over-the-counter (OTC) solutions, artificial tears, gels or scrubs and cannot discontinue these medications for the duration of the trial;
k) Use tear substitutes or any other ophthalmic medications 2 hours prior to the start of the study;
l) Have used Restasis® within 30 days of the study visit;
m) Have taken, in the past 30 days, any medication known to cause ocular drying that has not been used on a stable dosing regimen for 30 days prior to the study visit.

Qualified subjects received a single drop of AC-111 in one eye and a single drop of vehicle (i.e. no HPMC component) in the neighboring eye. TFBUT was assessed pre and post CAE exposure.

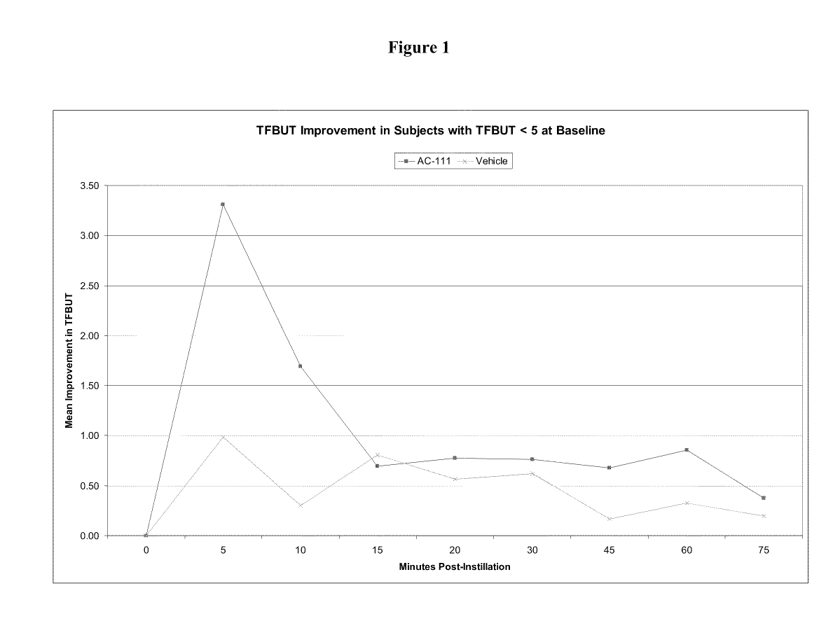

The results are shown in FIG. 1. As shown in FIG. 1, AC-111 was more effective in improving tear film stability post-CAE exposure (i.e., increasing TFBUT) over a 75 minute time period as compared to vehicle control. In particular, the increase in TFBUT was statistically significant at 5 minutes and up to 10 minutes post-CAE exposure.

The TFBUT may be used to derive an ocular protection index (OPI) (Nally L, Ousler G W, Abelson M B. Ocular discomfort and tear film break-up time in dry eye 25 patients: a correlation. IOVS 2000 41; 4 (ARVO Abstract): 1436.), which is obtained by dividing the TFBUT by the time in seconds between blinks (the inter-blink interval, or “IBI”). An OPI of 1 or more than 1 (that is, the TFBUT is greater than or equal to the IBI) indicates a tear-protected ocular surface, with minimized signs or symptoms of dry eye. An OPI of less than 1 (that is, the TFBUT is less than the IBI) indicates an unprotected ocular surface, with exacerbated signs or symptoms of dry eye.

### Example 4

### Assessment of the effect of a 0.8% HPMC-based Artificial Tear Solution on Clinical Signs of Dry Eye: Corneal Staining

The following study describes a double-masked, randomized, single-center study (N=45, 15 per treatment group) evaluating the effect of a 0.30% Ketorolac/0.80% HPMC formulation, a 0.30% Ketorolac alone formulation, and vehicle alone (i.e., no ketorolac, no HPMC component) on the clinical signs of dry eye, in particular corneal staining, induced by three repeated 15-minute challenges in the Controlled Adverse Environment (CAE) Model followed by a 20 minute visual tasking challenge (television watching). The total duration of the study was 7 days.

Inclusion Criteria: Participating subjects (N=45) were required to meet all of the following requirements to be eligible for enrollment into the study.

1) Be 18 years of age or older;
2) Be able and willing to follow instructions, including participation in study assessments, and can be present for the required study visits for the duration of the study;
1) Have a corrected visual acuity logMar +0.7 (ETDRS) or better in each eye;
2) Have a reported history of dry eye in each eye;
3) Have a tear film break up severity score of 2 or greater in both eyes, as graded by the clinical during Visit 1 pre-CAE tear film break up video assessments;
4) Have a history of use of or desire to use an eye drop for dry eye symptoms within the past 6 months;
5) Have a ≧1+ staining score in at least one eye prior to exposure to the CAE at Visit 1;
6) Have a Cochet Bonnet score greater than or equal to 55 mm in both eyes at both visits;
7) (If female and of childbearing potential) Not be pregnant, nursing, or planning a pregnancy. Women of childbearing potential are required to have a negative urine pregnancy test at the screening and exit visits and agree to use an acceptable method of contraception for the duration of the study.

Exclusion Criteria: Subjects were excluded from participating in the study if any of the following criteria were met:

1) Have contraindications to the use of the study medication (s);
2) Have known allergy or sensitivity to the study medication (s) or their components;
3) Have anterior blepharitis, which is deemed clinically significant and/or likely to interfere with study parameters in the opinion of the investigator;
4) Be diagnosed with an on-going ocular infection (bacterial, viral, or fungal), or active ocular inflammation (e.g., follicular conjunctivitis);
5) Wear contact lenses within 1 week of Visit 1 or throughout the course of the study;
6) Have previously had laser in situ keratomileusis (LASIK) surgery;
7) Be currently taking any topical ophthalmic prescription or over-the-counter (OTC) solutions, artificial tears, gels or scrubs and cannot discontinue these medications for the duration of the trial;
8) Have used Restasis® within 30 days of Visit 1;
9) Have a systemic disease, or uncontrolled medical condition, that in the opinion of the investigator could interfere with study measurements or subject compliance;
10) Be currently taking (at Visit 1) any medication known to cause ocular drying that has not been used on a stable dosing regimen for 30 days prior to Visit 1;
11) Be currently pregnant, nursing, or planning a pregnancy;
12) (For women of childbearing potential) Be unwilling to submit a urine sample for a pregnancy test at Visit 1 and at exit visit.

Baseline levels of corneal staining were obtained from the qualified subjects prior to CAE exposure. Qualified subjects were then randomized prior to CAE exposure into one of the three treatment arms, and received a dose of the randomized treatment in both eyes immediately prior to CAE exposure. Subjects were then exposed to the first 15 minute CAE challenge interval, and corneal staining was assessed. This process was repeated for the second and third CAE challenge intervals. Bilateral dosage was repeated prior to the second CAE 15 minute challenge, and corneal staining was again assessed. This process was repeated for the third CAE challenge interval.

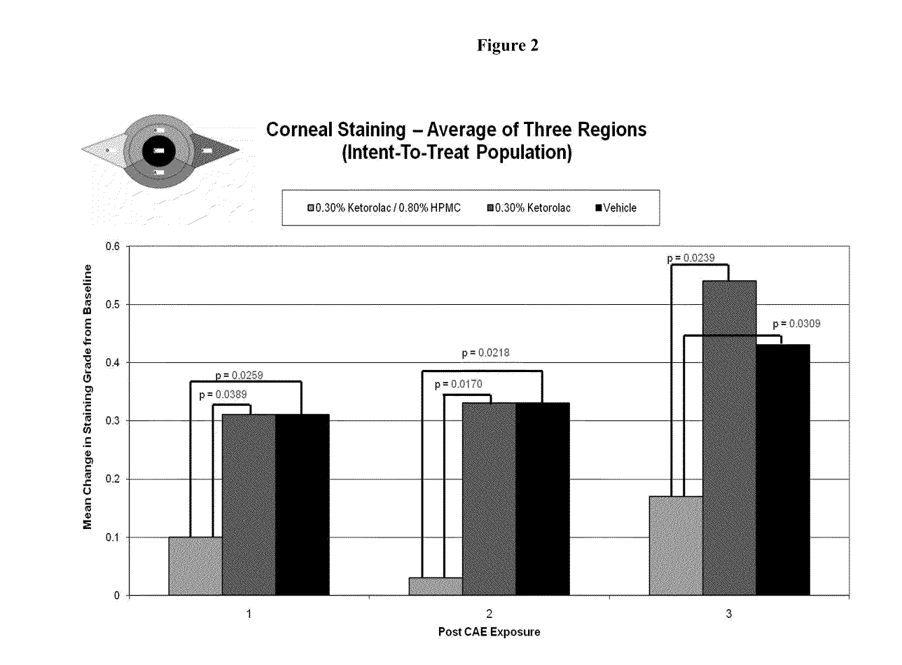

The results of the study are shown in FIG. 2. As shown in FIG. 2, the combined 0.3% ketorolac/0.8% HPMC formulation reduced corneal staining in three different regions of the eye, whereas both the 0.3% ketorolac alone formulation and vehicle alone formulation (i.e., no ketorolac, no HPMC component), had no effect. The results were statistically significant for all three regions measured. Thus, the reduction in corneal staining was clearly attributed to the 0.8% HPMC component (i.e., AC-111) of the combined 0.3% ketorolac/0.8% HPMC formulation, indicating that AC-111 is efficacious in treating the signs of dry eye. Moreover, AC-111 was capable of significantly reducing corneal staining after just 1 dose and showed an accumulated benefit over the course of a day after just 3 doses. Such immediate efficacy has not been previously achieved by commercially tear formulations currently available on the market.

### Example 5

### Clinical Efficacy of a 0.88% HMPC-based Artificial Tear Solution on Improving Clinical Symptoms of Dry Eye

The following study describes a double-masked, randomized, single-center, environmental study evaluating the effect of a 0.8% HPMC formulation 0.8% HPMC ophthalmic solution (8.0 mg/ml Hypromellose, USP Type 2910 (Methocel® E4M Premium—Hydroxypropyl methylcellulose); 8.0 mg/ml sodium chloride, USP; 0.15 mg/ml edetate sodium, USP; Sodium hydroxide, 5 N or Hydrochloric Acid, 5 N to adjust pH to 7.4; and purified water, USP (QS to 1 ml)), referred to herein as “AC-111”, used QID for 6 weeks in patients diagnosed with dry eye as compared to a vehicle control (i.e., no HPMC component).

Inclusion Criteria: Subjects who participated in the study (N=20 per treatment group) were required to meet all of the following requirements to be eligible for enrollment into the study:

1) Be 18 years of age or older;
2) Be able and willing to follow instructions, including participation in study assessments, and can be present for the required study visits for the duration of the study;
3) Have a diagnosis of mild to moderate dry eye;
4) Have a history of use of or desire to use an eye drop for dry eye symptoms within the past 6 months;
5) Have a best corrected visual acuity (BCVA) of +0.7 or better assessed by Early Treatment of Diabetic Retinopathy Study (ETDRS) scale in both eyes at Visit 1;
6) (If female and of childbearing potential) Not be pregnant, nursing, or planning a pregnancy. Women of childbearing potential are required to have a negative urine pregnancy test at the screening and exit visits and agree to use an acceptable method of contraception for the duration of the study.

Exclusion Criteria: Subjects were excluded from the study if they met any of the following criteria:

1) Have anterior blepharitis, which is deemed clinically significant and/or likely to interfere with study parameters in the opinion of the investigator;
2) Be diagnosed with an on-going ocular infection (bacterial, viral, or fungal), or active ocular inflammation (e.g., follicular conjunctivitis);
3) Wear contact lenses within 1 week of Visit 1 or throughout the course of the study;
4) Have used any eye drop within 4 hours of any of the visits;
5) Have previously had laser in situ keratomileusis (LASIK) surgery;
6) Be currently taking any topical ophthalmic prescription or over-the-counter (OTC) solutions, artificial tears, gels or scrubs and cannot discontinue these medications for the duration of the trial after randomization;
7) Have used Restasis® within 30 days of Visit 1;
8) Have a systemic disease, or uncontrolled medical condition, that in the opinion of the investigator could interfere with study measurements or subject compliance;
9) Be currently taking (at Visit 1) any medication known to cause ocular drying that has not been used on a stable dosing regimen for 30 days prior to Visit 1;
10) Be currently pregnant, nursing, or planning a pregnancy;
11) (For women of childbearing potential) Be unwilling to submit a urine sample for a pregnancy test at Visit 1 and at exit visit;
12) Have received another experimental drug or device within 30 days of visit 1.

Subjects received QID dosing of AC-111 in one eye, and of vehicle (i.e., no HPMC component) in the other eye (N=20 per treatment group).

Each subject kept a daily diary in which they assessed and recorded their level of ocular discomfort, in particular the level of burning, dryness, grittiness and stinging in each eye at four different timepoints throughout the day (morning, afternoon, evening, bedtime) and upon awakening each following morning. At each timepoint, subjects were asked to grade the level burning, dryness, grittiness and stinging in each eye based on a scale of zero (least severe) to 5 (most severe).

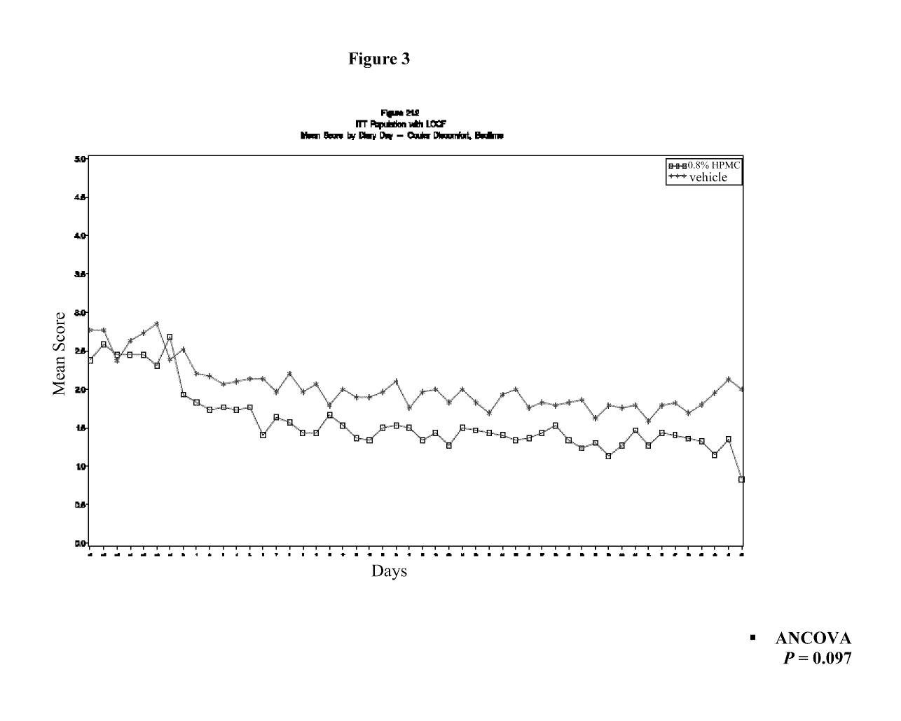

As shown in FIG. 3, AC-111 was more effective than the vehicle control in reducing ocular discomfort in dry-eye subjects over the six week period days (FIG. 3). AC-111 was also effective at reducing ocular discomfort in dry eye subjects at specific timepoints throughout the day (morning, afternoon, evening, bedtime, the following morning) over the six-week period (see FIG. 4, representing the mean scores at each timepoint over the six week time period). Moreover, AC-111 was capable of reducing the amount of ocular discomfort after immediate use (i.e., day 1) and continued to improve benefits with continued use over the 6 week period. Such efficacy has not been previously shown by commercial tear formulations currently available on the market.

### Example 6

### Analysis of Viscosity of Artificial Tear Solutions on Clinical Symptoms of Dry Eye

HMPC-based ophthalmic solutions were manufactured in accordance with the methods described herein at varying mid to high viscosities, 70 cpi, 90 cpi and 120 cpi, to identify the viscosity that performed the best in the ocular setting (i.e., coated the ocular surface the best without caking, clumping, or other side effects).

Each of these test formulations (70 cpi, 90 cpi and 120 cpi) were combined with 0.25% ketorolac and were evaluated for efficacy in treating clinical signs of dry eye (TFBUT and OPI) in subjects exposed to the CAE chamber, as previously described in Example 3. Side-effects of the test formulations, including lid caking, blurring and drop comfort, were also subjectively evaluated by each subject.

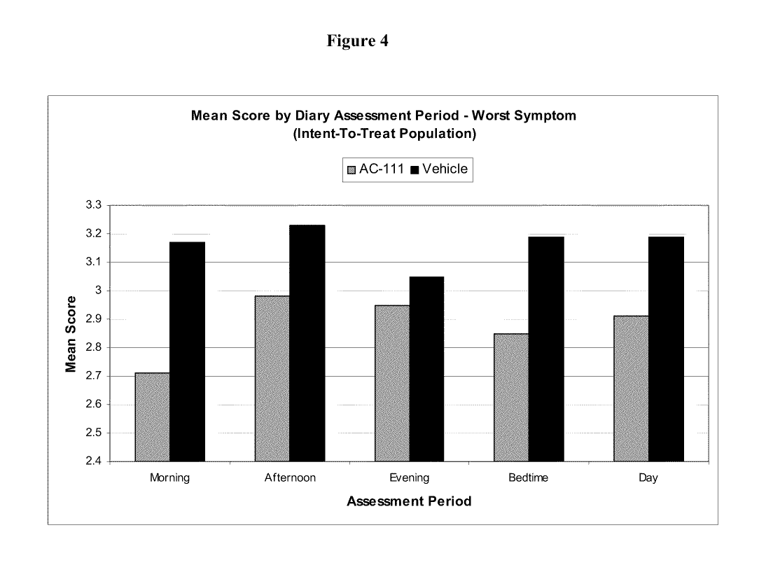

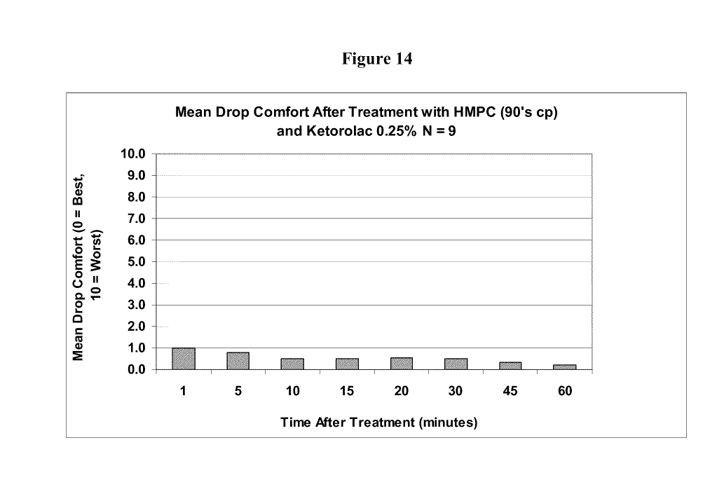

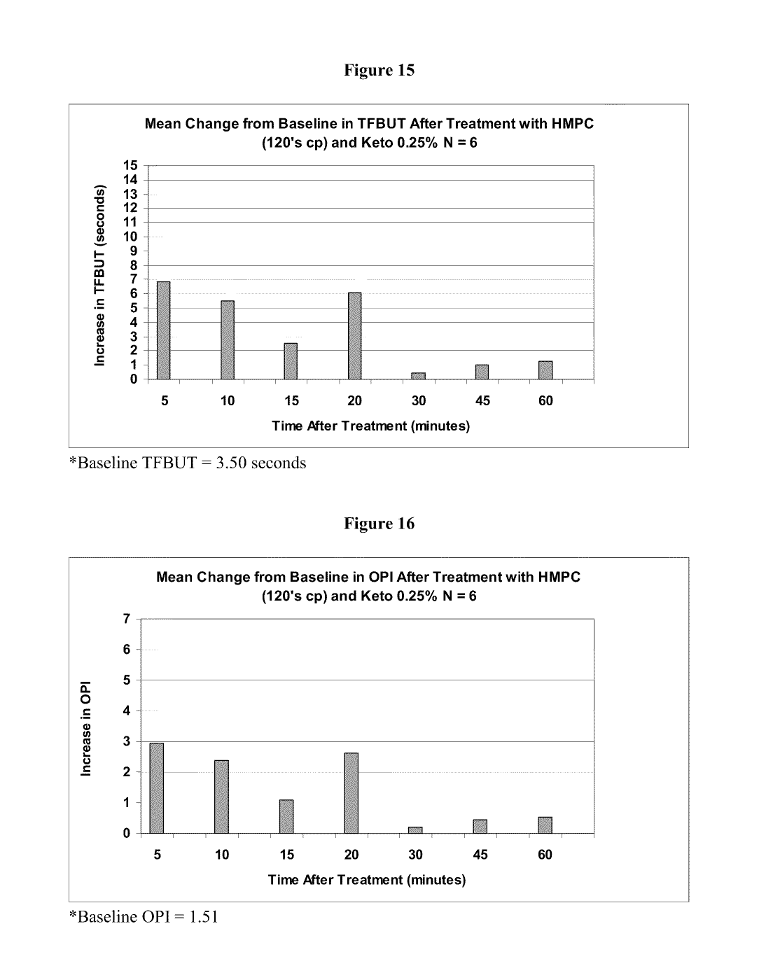

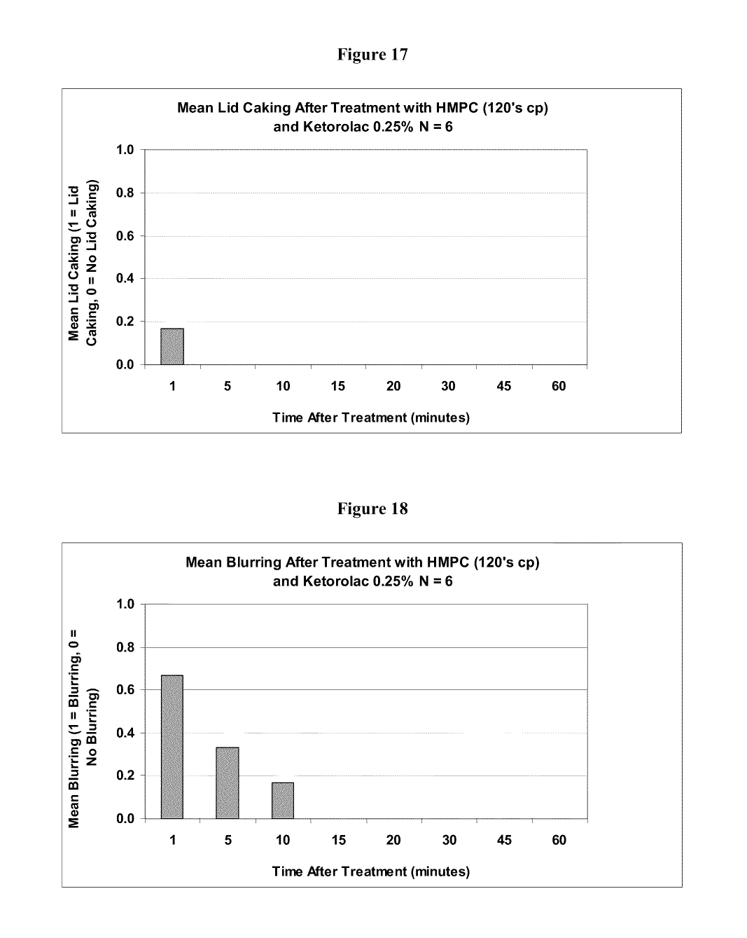

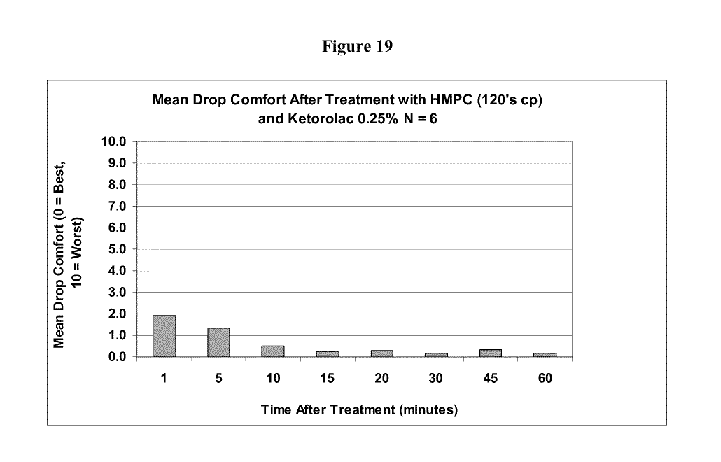

The results for the 70 cpi formulation are shown in FIGS. 5-9. The results for the 90 cpi formulation are shown in FIGS. 10-14. The results for the 120 cpi formulation are shown in FIGS. 15-19.

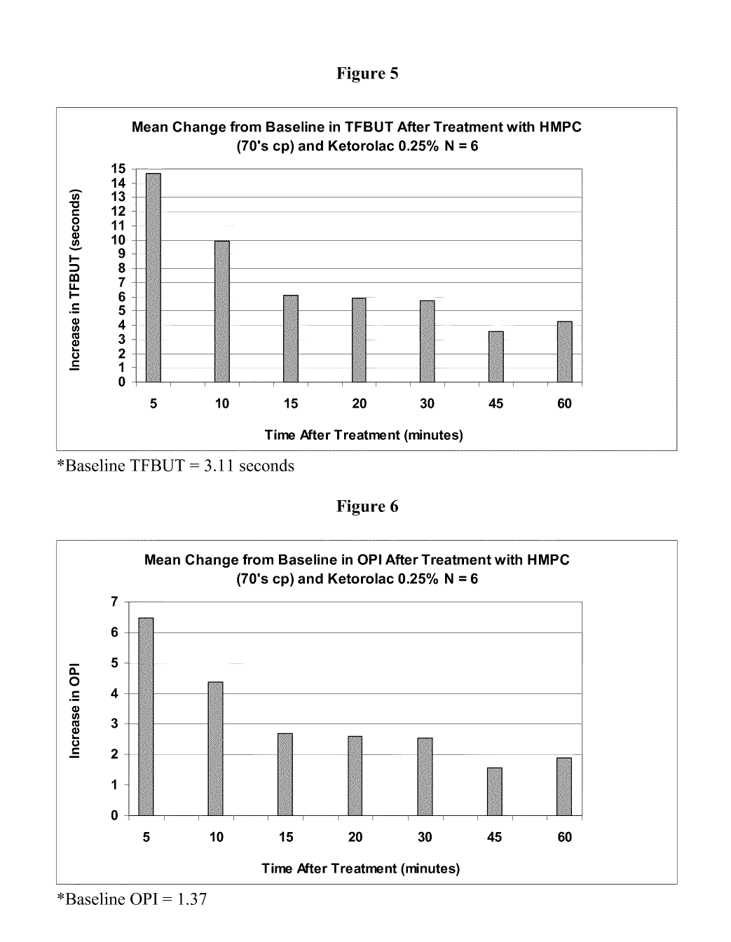

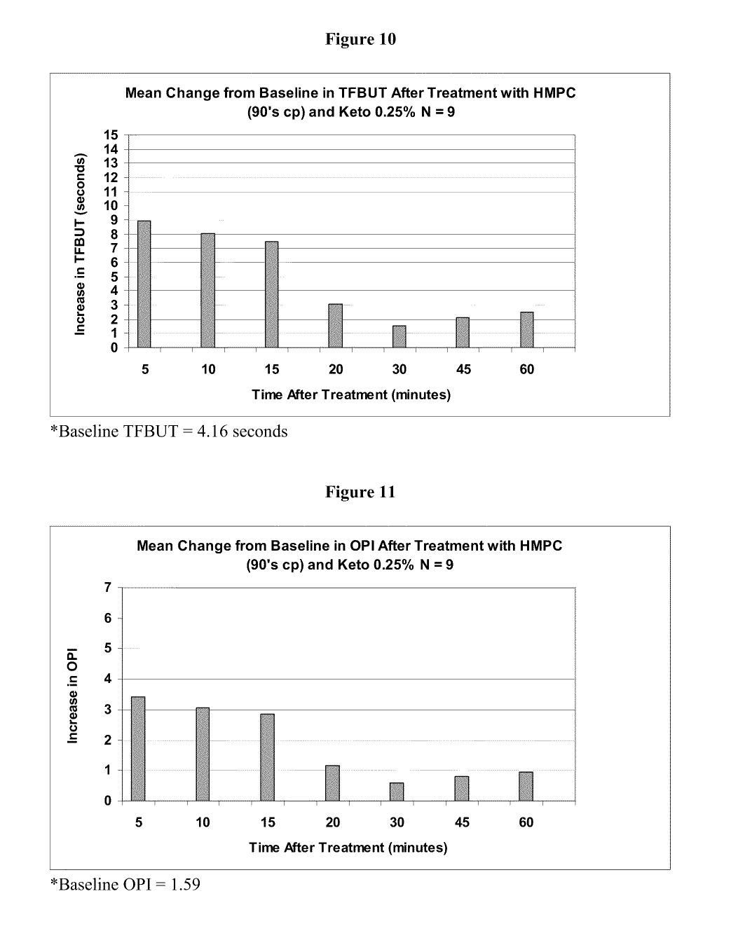

Surprisingly, the test formulation with the lowest viscosity (i.e., 70 cpi), was the most effective in treating the clinical signs of dry eye. As shown in FIGS. 5 and 6, the 70 cpi formulation had a greater effect on improving tear film integrity as evidenced by a greater increase in TFBUT and OPI as compared to the 90 cpi formulation (see FIGS. 10 and 11) and the 120 cpi formulation (see FIGS. 15 and 16). These results were unexpected as one skilled in the art would expect that the test formulation with the highest viscosity (i.e., 120 cpi) would be more effective in improving tear film integrity.

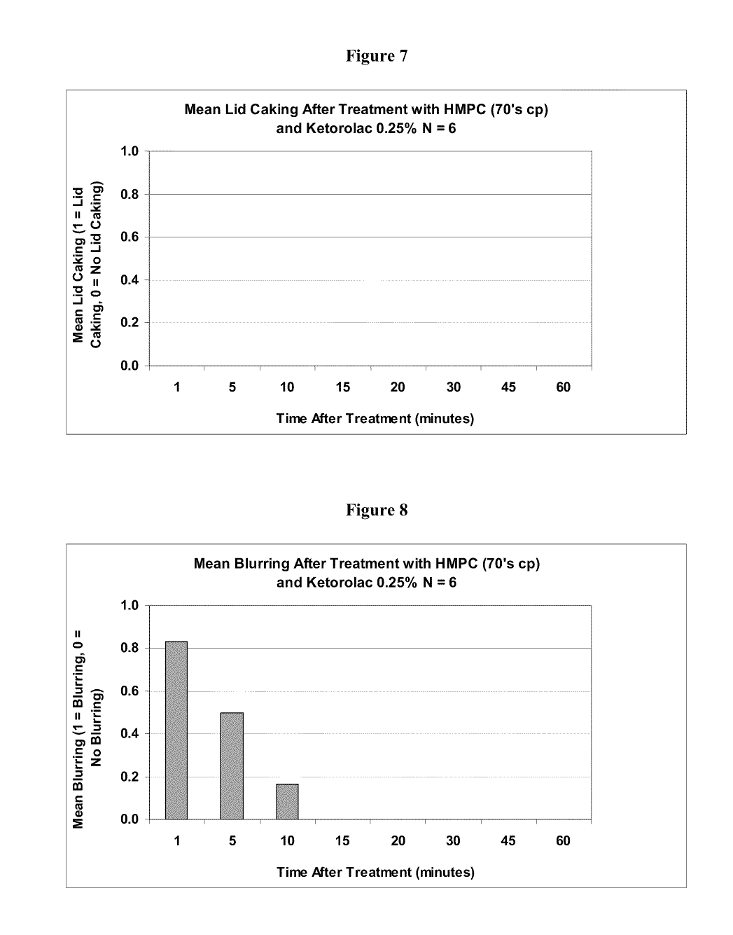

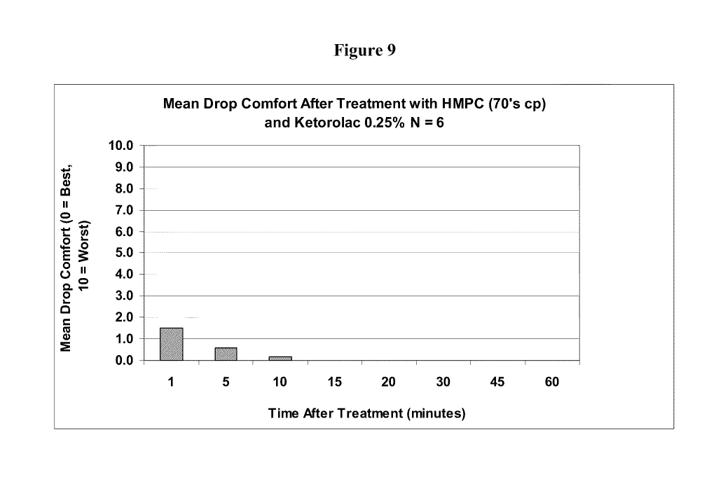

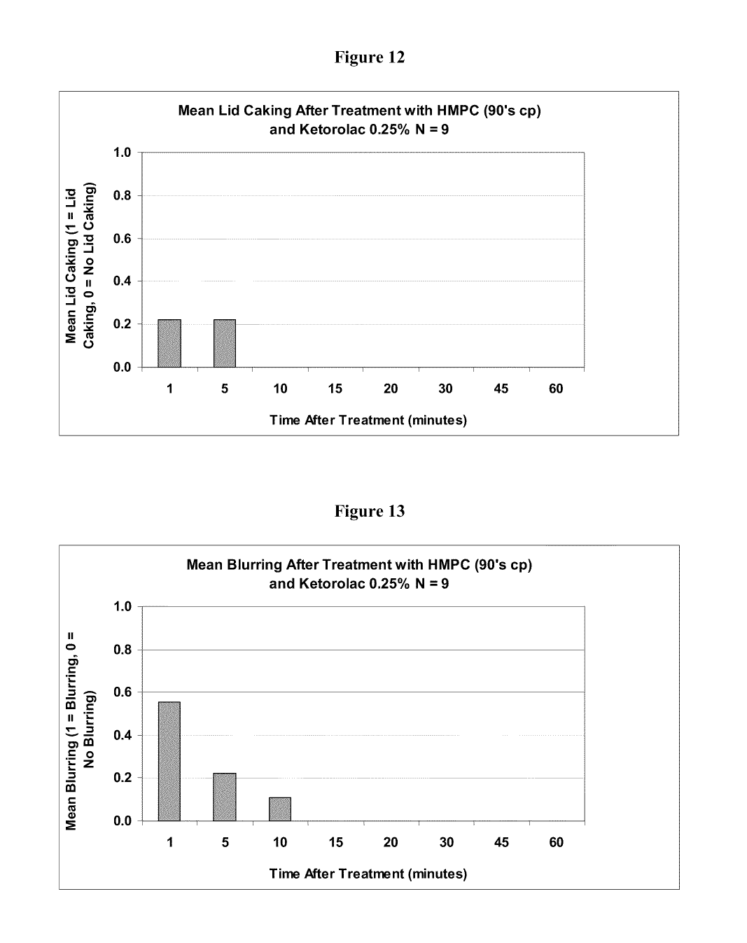

The 70 cpi test formulation also performed better in the ocular setting than the 90 cpi and 120 cpi test formulations. As shown in FIG. 7, the 70 cpi test formulation caused no lid caking, whereas the 90 cpi formulation and the 120 cpi formulation each caused lid caking for up to 5 minutes post-instillation (see FIGS. 12 and 17, respectively). Likewise, the 70 cpi test formulation was also more comfortable in the eye than the 90 cpi formulation and the 120 cpi formulation (see FIGS. 9, 14 and 19) over a 60 minute time period.

These results demonstrate that there is an optimal viscosity range which coats the ocular surface better than prior artificial tear solutions known in the art (i.e., no clumping or caking), leading to an increased retention time on the ocular surface over prior artificial tear formulations of varying viscosity, thereby enhancing the integrity of the tear film and providing protection of the ocular surface (e.g., as evidenced by increased tear film break-up time and ocular protection index). As demonstrated to herein, the 70 cpi formulation was more effective at treating the clinical signs of dry eye, and performed better in the ocular setting (i.e., better coating of the ocular surface and more comfortable). The formulations having a viscosity of 90 cpi or greater were less effective in treating the clinical signs of dry eye, caused visual blurring, and were less comfortable in the eye. These results demonstrate that an artificial tear solution having a viscosity ranging between 70 cpi to less than 90 cpi (e.g., 70 cpi to 88 cpi), is optimal for coating the ocular surface and thereby treating dry eye.

### REFERENCES

All publications and patents mentioned herein are hereby incorporated by reference in their entireties as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

We claim:

1. A method of making an ophthalmic formulation for instillation into the eye comprising: heating a solution base comprising water to at least 85° C.; mixing the solution base at a rate of at least 10 rotations per minute; adding hypromellose at a rate of at least 0.1 gm/min until a target viscosity is achieved; wherein the target viscosity is from about 70 to 88 cpi.

2. The method of claim 1 further comprising adding three or more tonicity agents selected from the group consisting of NaCl, KCl, ZnCl₂, CaCl₂, and MgCl₂.

3. The method of claim 2 wherein the tonicity agents are added in a ratio that provides an osmolality range of about 260 to about 300 mOsm/kg.

4. The method of claim 1 further comprising adding a buffer system selected from the group consisting of borate buffers, phosphate buffers, citrate buffers, and combinations and mixtures thereof.

5. An ophthalmic formulation for instillation into the eye manufactured by a method comprising:
a. heating a solution base comprising water to at least 85° C.;
b. mixing the solution base at a rate of at least 10 rotations per minute;
c. adding hypromellose at a rate of at least 0.1 gm/min until a target viscosity is achieved, wherein the target viscosity is from about 70 to 88 cpi;
d. adding three or more tonicity agents selected from the group consisting of NaCl, KCl, ZnCl₂, CaCl₂, and MgCl₂, wherein the three or more tonicity agents are added in a ratio that provides an osmolality range of about 260 to about 300 mOsm/kg; and
e. adding a buffer system selected from the group consisting of borate buffers, phosphate buffers, citrate buffers, and combinations and mixtures thereof.

6. An ophthalmic formulation comprising 8.0 mg/ml Hypromellose, 8.0 mg/ml sodium chloride, and 0.15 mg/ml disodium edetate, wherein the pH of the formulation is 7.4 and the viscosity of the formulation is 70-88 cpi.

7. An ophthalmic formulation comprising 8.0 mg/ml Hypromellose, 10 mg/ml glycerin, 4.8 mg/ml sodium chloride, 0.8 mg/ml potassium chloride, 0.08 mg/ml calcium chloride, 0.05 mg/ml magnesium chloride, and 1.7 mg/ml sodium citrate, wherein the pH of the formulation is 7.4 and the viscosity of the formulation is 70-88 cpi.

8. The ophthalmic formulation of claim 7, wherein the formulation is for unit dose administration.

9. An ophthalmic formulation comprising 8.0 mg/ml Hypromellose, 10 mg/ml glycerin, 4.8 mg/ml sodium chloride, 0.8 mg/ml potassium chloride, 0.08 mg/ml calcium chloride, 0.05 mg/ml magnesium chloride, 5.0 mg/ml boric acid, 1.0 mg/ml sorbic acid, and 1.0 mg/ml disodium edetate, wherein the pH of the formulation is 7.4 and the viscosity of the formulation is 70-88 cpi.

10. The ophthalmic formulation of claim 9, wherein the formulation is for multi-dose administration.

11. A method for treating the signs and symptoms of dry eye comprising topically administering to the eye of a subject in need thereof the ophthalmic formulation of any one of claims 1, 6, 7 or 9.
